SEQUENCE LISTING (53 sequences)

SEQ ID NO: 1 - RNA, 39 nt, Artificial sequence
  Description of the artificial sequence: The amino acid sequence for another protease substrate according to the present invention
gggaguugauaacaggcucaaugagccugcucggucaac

SEQ ID NO: 2 - RNA, 39 nt, Artificial sequence
  Description of the artificial sequence: Rev-Protein binding candidate sequence
gggaguugauagcaggcucaaugagccugaguucccaac

SEQ ID NO: 3 - RNA, 39 nt, Artificial sequence
  Description of the artificial sequence: Rev-Protein binding candidate sequence
gggaguugauaucaggcucaaugagccuggucgaccaac

SEQ ID NO: 4 - RNA, 39 nt, Artificial sequence
  Description of the artificial sequence: Rev-Protein binding candidate sequence
gggaguugauaccaggcucaaugagccugcaaagucaac

SEQ ID NO: 5 - RNA, 37 nt, Artificial sequence
  Description of the artificial sequence: Rev-Protein binding candidate sequence
gggagguggacuccagcuucggcuguugagauacacc

SEQ ID NO: 6 - RNA, 39 nt, Artificial sequence
  Description of the artificial sequence: Rev-Protein binding candidate sequence
gggaguugguaccaggcucaaugagccugaaagcucaac

SEQ ID NO: 7 - RNA, 39 nt, Artificial sequence
  Description of the artificial sequence: Rev-Protein binding candidate sequence
gggaguugcuaccaggcucaaugagccugguuaaacaac

SEQ ID NO: 8 - RNA, 39 nt, Artificial sequence
  Description of the artificial sequence: Rev-Protein binding candidate sequence
gggaguuguuaccaggcucaaugagccugcgcgcgcaac

SEQ ID NO: 9 - RNA, 39 nt, Artificial sequence
  Description of the artificial sequence: Rev-Protein binding candidate sequence
gggaguuguaaccaggcucaaugagccuguauauacaac

SEQ ID NO: 10 - RNA, 39 nt, Artificial sequence
  Description of the artificial sequence: Rev-Protein binding candidate sequence
gggaguugugaccaggcucaaugagccugagaauccaac

SEQ ID NO: 11 - RNA, 39 nt, Artificial sequence
  Description of the artificial sequence: Rev-Protein binding candidate sequence
gggaguugucaccaggcucaaugagccugccuggacaac

SEQ ID NO: 12 - RNA, 39 nt, Artificial sequence
  Description of the artificial sequence: Rev-Protein binding candidate sequence
gggaguuggcaucaggcucaaugagccuguggacacaac

SEQ ID NO: 13 - RNA, 39 nt, Artificial sequence
  Description of the artificial sequence: Rev-Protein binding candidate sequence
gggaguuggacucaggcucaaugagccuggaaaaacaac

SEQ ID NO: 14 - RNA, 39 nt, Artificial sequence
  Description of the artificial sequence: Rev-Protein binding candidate sequence
gggaguuggaaucaggcucaaugagccugaggggacaac

SEQ ID NO: 15 - RNA, 39 nt, Artificial sequence
  Description of the artificial sequence: Rev-Protein binding candidate sequence
gggaguuggccucaggcucaaugagccugcuuuuccaac

SEQ ID NO: 16 - RNA, 39 nt, Artificial sequence
  Description of the artificial sequence: Rev-Protein binding candidate sequence
gggaguuggaaucaggcucaaugagccuguccccucaac

SEQ ID NO: 17 - RNA, 39 nt, Artificial sequence
  Description of the artificial sequence: Rev-Protein binding candidate sequence
gggaguuggccucaggcucaaugagccugaccccacaac

SEQ ID NO: 18 - RNA, 39 nt, Artificial sequence
  Description of the artificial sequence: Rev-Protein binding candidate sequence
gggaguuggggucaggcucaaugagccugguuuugcaac

SEQ ID NO: 19 - RNA, 39 nt, Artificial sequence
  Description of the artificial sequence: Rev-Protein binding candidate sequence
gggaguugguuucaggcucaaugagccugcgggggcaac

SEQ ID NO: 20 - RNA, 39 nt, Artificial sequence
  Description of the artificial sequence: Rev-Protein binding candidate sequence
gggaguugaauucaggcucaaugagccuguaaaaucaac

SEQ ID NO: 21 - RNA, 39 nt, Artificial sequence
  Description of the artificial sequence: Rev-Protein binding candidate sequence
gggaguugaacccaggcucaaugagccugauauaucaac

SEQ ID NO: 22 - RNA, 39 nt, Artificial sequence
  Description of the artificial sequence: Rev-Protein binding candidate sequence
gggaguugaacacaggcucaaugagccuguauauacaac

SEQ ID NO: 23 - RNA, 39 nt, Artificial sequence
  Description of the artificial sequence: Rev-Protein binding candidate sequence
gggaguugaaggcaggcucaaugagccugaaauuucaac

SEQ ID NO: 24 - RNA, 39 nt, Artificial sequence
  Description of the artificial sequence: Rev-Protein binding candidate sequence
gggaguugaagucaggcucaaugagccuguuuaaacaac

SEQ ID NO: 25 - RNA, 39 nt, Artificial sequence
  Description of the artificial sequence: Rev-Protein binding candidate sequence
gggaguugaguccaggcucaaugagccugaauuaacaac

SEQ ID NO: 26 - RNA, 39 nt, Artificial sequence
  Description of the artificial sequence: Rev-Protein binding candidate sequence
gggaguuguuuccaggcucaaugagccuguuaauucaac

SEQ ID NO: 27 - RNA, 39 nt, Artificial sequence
  Description of the artificial sequence: Rev-Protein binding candidate sequence
gggaguugggggcaggcucaaugagccugcccaaacaac

SEQ ID NO: 28 - RNA, 39 nt, Artificial sequence
  Description of the artificial sequence: Rev-Protein binding candidate sequence
gggaguuggcaacaggcucaaugagccugcacagucaac

SEQ ID NO: 29 - RNA, 39 nt, Artificial sequence
  Description of the artificial sequence: Rev-Protein binding candidate sequence
gggaguugaccccaggcucaaugagccuggugcagcaac

SEQ ID NO: 30 - RNA, 39 nt, Artificial sequence
  Description of the artificial sequence: Rev-Protein binding candidate sequence
gggaguugcacacaggcucaaugagccuggucagccaac

SEQ ID NO: 31 - RNA, 39 nt, Artificial sequence
  Description of the artificial sequence: Rev-Protein binding candidate sequence
gggaguugcauacaggcucaaugagccugcaguuacaac

SEQ ID NO: 32 - RNA, 39 nt, Artificial sequence
  Description of the artificial sequence: Rev-Protein binding candidate sequence
gggaguugugagcaggcucaaugagccugggcagucaac

SEQ ID NO: 33 - RNA, 39 nt, Artificial sequence
  Description of the artificial sequence: Rev-Protein binding candidate sequence
gggaguuggguccaggcucaaugagccugucaacucaac

SEQ ID NO: 34 - RNA, 39 nt, Artificial sequence
  Description of the artificial sequence: Rev-Protein binding candidate sequence
gggaguugcgaucaggcucaaugagccugacuaggcaac

SEQ ID NO: 35 - RNA, 39 nt, Artificial sequence
  Description of the artificial sequence: Rev-Protein binding candidate sequence
gggaguugaauccaggcucaaugagccuguugcaccaac

SEQ ID NO: 36 - RNA, 39 nt, Artificial sequence
  Description of the artificial sequence: Rev-Protein binding candidate sequence
gggaguugucggcaggcucaaugagccugacguaccaac

SEQ ID NO: 37 - RNA, 39 nt, Artificial sequence
  Description of the artificial sequence: Rev-Protein binding candidate sequence
gggaguuguccgcaggcucaaugagccugugguaacaac

SEQ ID NO: 38 - RNA, 39 nt, Artificial sequence
  Description of the artificial sequence: Rev-Protein binding candidate sequence
gggaguugagaacaggcucaaugagccugcuccgacaac

SEQ ID NO: 39 - RNA, 39 nt, Artificial sequence
  Description of the artificial sequence: Rev-Protein binding candidate sequence
gggaguugccuccaggcucaaugagccugaccucgcaac

SEQ ID NO: 40 - RNA, 39 nt, Artificial sequence
  Description of the artificial sequence: Rev-Protein binding candidate sequence
gggaguugcccgcaggcucaaugagccuggcagcccaac

SEQ ID NO: 41 - RNA, 39 nt, Artificial sequence
  Description of the artificial sequence: Rev-Protein binding candidate sequence
gggaguuggcgccaggcucaaugagccugucgaagcaac

SEQ ID NO: 42 - RNA, 39 nt, Artificial sequence
  Description of the artificial sequence: Rev-Protein binding candidate sequence
gggaguugcgcgcaggcucaaugagccugucacaccaac

SEQ ID NO: 43 - RNA, 39 nt, Artificial sequence
  Description of the artificial sequence: Rev-Protein binding candidate sequence
gggaguugcgaacaggcucaaugagccugaaaaaacaac

SEQ ID NO: 44 - RNA, 39 nt, Artificial sequence
  Description of the artificial sequence: Rev-Protein binding candidate sequence
gggaguugucuucaggcucaaugagccuguuuuuucaac

SEQ ID NO: 45 - RNA, 39 nt, Artificial sequence
  Description of the artificial sequence: Rev-Protein binding candidate sequence
gggaguugggagcaggcucaaugagccugggggggcaac

SEQ ID NO: 46 - RNA, 39 nt, Artificial sequence
  Description of the artificial sequence: Rev-Protein binding candidate sequence
gggaguuggccucaggcucaaugagccugcccccccaac

SEQ ID NO: 47 - RNA, 39 nt, Artificial sequence
  Description of the artificial sequence: Rev-Protein binding candidate sequence
gggaguuggaugcaggcucaaugagccugaaauggcaac

SEQ ID NO: 48 - RNA, 39 nt, Artificial sequence
  Description of the artificial sequence: Rev-Protein binding candidate sequence
gggaguugcccucaggcucaaugagccugaaauggcaac

SEQ ID NO: 49 - RNA, 39 nt, Artificial sequence
  Description of the artificial sequence: Rev-Protein binding candidate sequence
gggaguugcucucaggcucaaugagccugauauggcaac

SEQ ID NO: 50 - RNA, 39 nt, Artificial sequence
  Description of the artificial sequence: Rev-Protein binding candidate sequence
gggaguugcucgcaggcucaaugagccugaaaugacaac

SEQ ID NO: 51 - RNA, 56 nt, Artificial sequence
  : Description of the artificial sequence: Hammerhead ribozyme construct system 1
ggguccucugaugagcucugggcgcagcuucggcugacgguacuugcgaaacucgu

SEQ ID NO: 52 - RNA, 65 nt, Artificial sequence
  Description of the artificial sequence: Hammerhead ribozyme construct system 1
gggacucgcuucggcuugagauaccaaaaaggguccucugaugaggccguuaggccgaaacucgu

SEQ ID NO: 53 - RNA, 13 nt, Artificial sequence
acgagucaggauu

(19) United States
(12) Patent Application Publication
(10) Pub. No.: US 2005/0202423 A1
(43) Pub. Date: Sep. 15, 2005

(54) Method for identifying compounds or lead structures against rna target motifs and rna/protein interactions

(76) Inventors: Andres Jenne, Prien am Chiemsee (DE); Michael Blind, Munchen (DE); Michael Famulok, Bonn (DE); Seyed Hani Najafi-Shoushtari, Bonn (DE); Jorg Hartig, Bonn (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/432,560

(22) PCT Filed: Nov. 21, 2001
(86) PCT No.: PCT/EP01/13568

§ 371 Date: Mar. 14, 2005

(30) Foreign Application Priority Data
Nov. 22, 2000 (DE) 100 57 853.5
Sep. 10, 2001 (DE) 101 44 355.2

(51) Int. Cl. C12Q 1/68; C07H 21/02; C12P 19/34

(52) U.S. Cl. 435/6; 536/25.32; 435/91.2

(57) ABSTRACT

Disclosed is a method for identifying compounds (lead structures) which specifically bind to (a) desired RNA target motif and can inhibit or eliminate the function thereof or (b) suppress a compound associated with a desired RNA target motif and can thereby inhibit or eliminate the function thereof. The inventive method is based on the attachment of a ligand (=a compound to be identified) to a RNA target motif which is coupled to a modified ribozyme so that the ribozyme is transformed into an active or inactive conformation resulting in the cleaving of a signal-giving ribozyme substrate. The identified compounds enabling modification of the cellular function of the RNA target motifs enable specific medicaments to be produced. The invention also relates to a polynucleotide comprising a hammerhead ribozyme and an aptamer for a target molecule. The base pairing model of the polynucleotide, when the target molecule binds to the aptamer, is different from the base pairing model of the polynucleotide when the target molecule does not bind to the aptamer.

The present invention relates to a procedure for the identification of compounds (lead structures) which either (a) specifically bind to a desired RNA-target motif and which can, as a result, inhibit or eliminate its function, or (b) displace a compound which is associated with a desired RNA-target motif and which can, as a result, inhibit or eliminate its function. The procedure in accordance with the invention is based on the binding of a ligand (=compound to be identified) to an RNA-target motif which is coupled to a modified ribozyme, so that the ribozyme is transformed into an active or inactive (or more or less active) conformation, resulting in a measurable signal, for example, by cleavage of a signaling ribozyme substrate. The compounds identified in this way serve as pharmaceutical lead substances for the production of drugs, as they can specifically influence the cellular function of an RNA-target motif or a compound which is associated with an RNA-target motif.

The present invention further relates to a polynucleotide comprising a hammerhead ribozyme and an aptamer, a biosensor comprising this polynucleotide, a procedure for identifying a compound which binds to a target molecule and the use of coumermycin, nosiheptide and patulin.

Ribonucleic acids have essential functions in a multitude of cellular processes, for example, protein synthesis, the regulation of gene expression and the processing of mRNAs. In addition, RNA molecules play a decisive role in the replication of retroviruses and therefore have an essential role in the manifestation of certain viral infections. For this reason, functional RNAs are an important class of “drug targets” in pharmaceutical research [D. S. Eggleston, C. D. Prescott & N. D. Pearson (Eds.), The Many Faces of RNA, Academic Press (1998) 83-96; Y. Tor et al., Chem. Biol. 5 (1998), R277-R283; N. D. Pearson & C. D. Prescott, Chem. Biol. 4 (1997), 409-414; T. Herrmann & E. Westhof, Curr. Opin. Biotech. 9 (1998), 66-73; M. Afshar et al., Curr. Opin. Biotech. 10 (1999), 59-63].

Small molecules which bind to RNA have been used for decades to treat bacterial infections [W. D. Wilson & K. Li, Curr. Med. Chem. 7 (2000), 73-98; Siegenthaler et al., Am. J. Med. 80 (1986), 2-14]. For example, aminoglycosides, such as neomycin B, paromomycin or streptomycin, are capable of inhibiting bacterial translation by binding to ribosomal RNA, giving an antibiotic effect [J. Woodcock et al., EMBO J. 10 (1991), 3099-3103; D. Moazed & H. F. Noller, Nature 327 (1987), 389-394]. Moreover, a series of aminoglycosides of the 2-desoxystreptamine family are capable of binding specifically to the RNA structural motif “RRE” of the HIV genome, resulting in inhibition of the replication of the HI virus [M. L. Zapp et al., Cell 74 (1993), 969-978; W. K. C. Park, et al., JACS 118 (1996), 10150-10155]. Although these and other antibiotics have been of therapeutic value for a long time, they have forfeited much of their efficacy in the interim, as an ever increasing number of pathogenic organisms have acquired resistance genes to the established drugs, which has caused current massive problems in the health system [J. Davies, Science 264 (1994), 375-382; H. C. Neu, Science 257 (1992), 1064-1073; J. Davies & G. D. Wright, Trends Microbiol. 5 (1997), 234-240].

For various reasons, functional RNA structures are extraordinarily well suited as target molecules for drugs. Just like proteins, RNAs and DNAs can form defined spatial structures and binding sites, where they can interact specifically with other molecules [O. C. Uhlenbeck et al., Cell 90 (1997), 833-840; S. M. Hecht, Bioorg. Med. Chem. 5 (1997), 1001-1248; C. S. Chow, Chem. Rev. 97 (1997), 1489-1513]. There is also less danger that retroviruses will develop resistance to RNA drugs, as the RNA target molecules are highly conserved and are initially present in the host cell in the unduplicated form [F. Hamy et al., PNAS USA 94 (1997), 3548-3553; M. Afshar et al., Curr. Opin. Biotech. 10 (1999), 59-63]. In addition, cellular mechanisms for the repair of RNA damages are totally unknown, even today [T. Herrmann & E. Westhof, Curr. Opin. Biotech. 9 (1998)].

Although there are a wide variety of reasons to use RNAs as therapeutic target molecules, drugs with RNA structures as targets are much less established than drugs at the DNA or protein level. There has therefore been great effort in modern biotechnology to develop antibiotics and antiviral drugs which are directed against defined RNA motifs [D. J. Ecker & R. H. Griffey, Drug Disc. Today 4 (1999), 420-429]. There are in principle two distinct strategies: (a) rational drug design and (b) combinatorial drug research.

In rational drug design, one attempts to predict the conformation of an RNA-drug complex on the basis of experimental structural and functional data. In particular, structural determination by NMR spectroscopy has contributed to the elucidation of the recognition of RNA by antibiotics at the molecular level [L. Jiang, et al., Chem. Biol. 4 (1997), 35-50; D. Fourmy, et al., Science 274 (1996), 1367-1371]. A very wide variety of computer programs have been used to exploit these data for rational drug design by calculating RNA-drug structural models [e.g. “Monte Carlo”: R. Rosenfeld et al., Annu. Rev. Biophys. Biomol. Struc. 24 (1995), 677-700; “DOCK”: Q. Chen et al., Biochemistry 36 (1997), 11402-11407; “Molecular Modeling”: T. Herrmann & E. Westhof, Curr. Opin. Biotech. 9 (1998), 66-73]. In comparison with this, there are relatively few approaches for determining the specificity of the RNA-drug interaction experimentally [M. Hendrix et al., J. Am. Chem. Soc. 119 (1997), 3641-3648; Y. Wang et al., Biochemistry 35 (1996), 12338-12346].

Although the latest studies have led to interesting results, the current state of the art only offers very limited possibilities for the specific design of RNA-binding active substances. To calculate RNA-active substance models, it is essential to understand the functional and structural principles of RNA-active substance recognition exactly, as these interactions occur at the levels of the primary, secondary and complex tertiary structure of the RNA [R. Schroeder et al., EMBO J. 19 (2000), 1-9; T. Herrmann & E. Westhof, Curr. Opin. Biotech. 9 (1998), 66-73]. In this context, the NMR structures of aminoglycoside-RNA complexes have provided valuable information on the principles of the interaction of RNA with small organic molecules [review: M. Afshar et al., Curr. Opin. Biotech. 10 (1999), 59-63]. In this way, it has, for example, been possible to employ the structure of the complex between ribosomal RNA and paromomycin as a basis to explain the binding properties of this antibiotic, its specificity for prokaryotic organisms and the development of resistance [D. Fourmy et al., Science 274 (1996), 1367-1371; S. C. Blanchard et al., Biochemistry 37 (1998), 7716-7724]. Aside from structural studies, specific functional analysis has contributed to the winning of insights into the molecular recognition of RNA-binding molecules. However, only a few research groups are responding to this challenge, as many of the known RNA-binding molecules are complex natural substances which are difficult to modify [W. K. C. Park et al., JACS 118 (1996), 10150-10155; H. Wang & Y. Tor, JACS 119 (1997), 8734-8735; J. H.-H. Tok & R. R. Rando, JACS 120 (1998), 8279-8280].

The growing recognition that RNA molecules can play a decisive role in the development of the clinical picture of a disease is accompanied by increasing efforts to develop empirical procedures to identify RNA-binding active substances [M. Afshar et al., Curr. Opin. Biotech. 10 (1999), 59-63]. At the moment, the most promising approach appears to be to use the technologies of combinatorial chemistry, in which libraries of candidate molecules are screened for active substances, using high-throughput techniques. There is increasing need today for high-throughput procedures, as these can be used to identify new lead structures to therapeutically relevant RNA structures—particularly those of known tertiary structure and regulatory significance. Lead structures of this sort are very useful as they can, either directly or indirectly, lead to the development of new therapeutically active substances. In addition, it would be extremely valuable for setting up data banks of RNA structure and RNA-function to identify as many low molecular weight substances as possible which bind specifically to defined RNA structures [P. Brion & E. Westhof, Annu. Rev. Biophys. Biomol. Struct. 26 (1997), 113-137].

Conventional standard methods for RNA analysis, such as gel retardation or filter binding experiments, assume kinetically stable RNA-ligand complexes for their detection and, for this reason alone, are unsuitable for the screening of large libraries of compounds on an industrial scale. What would be much more desirable would be to have a robust, non-radioactive and widely applicable method which would allow the rapid and reliable identification of new RNA-binding molecules.

As will be seen in the following review of the methods currently used for this purpose, an assay of this sort is unknown according to the present state of the art.

H.-Y. Mei et al. describe a procedure for the identification of inhibitors of the group I self-splicing intron in Pneumocystis carinii [H.-Y. Mei et al., NAR 24 (1996), 5051-5053]. Inhibitors of the group I intron splicing reaction are regarded as potential antibiotics for use in pulmonary infections which are caused by the fungus Pneumocystis carinii and which have a potentially fatal course for immune suppressed patients. Mei et al. carried out high-throughput screening and succeeded in identifying new inhibitory lead structures in a compound library of ca. 300,000 low molecular weight substances [H-Y Mei et al., Bioorg. Med. Chem. 5 (1997), 1185-1195]. However, the cleavage mechanism is specific for the group I intron, so that the procedure of Mei et al. is not transferable to other ribozyme reactions or RNA target structures, with the result that it can only be used in a narrow context. In addition, the procedure is based on radioactive detection and is also methodically demanding.

J. E. Arenas et al. have developed a screening procedure (“SCAN”) which allows the rapid identification of low molecular weight ligands to various RNA target sequences [J. E. Arenas et al., Nucleic Acids Symp. Series 41 (1999), 13-16]. With the help of this procedure, it was possible to isolate substances which bind to a specific regulatory RNA sequence, the so-called “epsilon-RNA” of the hepatitis B virus (HBV). Some of the isolated substances exhibited very promising antiviral properties in a cell-based HBV replication model. The procedure described by J. E. Arenas et al. exploits the different hybridization properties of free and ligand-bound RNA to complementary nucleic acid probes. Although this technique led to the identification of specific RNA-binding substances, the method is relatively demanding, as quantitative filtration steps must be performed to detect the RNA-ligand complexes. In addition, Arenas et al. used radioactively labeled RNAs for their high-throughput screening, evidently because other detection methods were not adequately sensitive.

In WO 98/18947A1, a procedure is described for the characterization and selection of RNA target molecules which bind to substances of therapeutic interest. The method described is also suitable for the identification of new active substances with potential pharmacological activity. RNA libraries are expressed for this purpose in living cells and brought into contact with the substance to be tested. However, the identification of an RNA-target molecule pair is based on the phenotypic analysis of living cells, so that this procedure is unsuitable for the rapid high-throughput in vitro screening of large substance libraries.

In PCT/GB99/01761, a fluorescence-based procedure is described for the in vitro identification of RNA-binding substances. It is necessary in this procedure to label both the target RNA structure being studied and an already known RNA ligand of this target structure with a fluorophore dye. If RNA and ligand are spatially separated from each other, the fluorescence of the fluorophore groups can be measured. In contrast, if a 1:1 complex between RNA and ligand is formed, the fluorescence is quenched. The identification of a new RNA-binding substance is based on the elimination of fluorescence quenching in the presence of an RNA-binding competitor, leading to a measurable signal. Although this procedure is suitable for the high-throughput screening of substance libraries, it is not generally applicable, as it always assumes that an RNA ligand has already been identified.

K. Hamasaki and R. Rando describe a fluorescence-based assay which can be used to examine specific interactions between RNA-binding substances and specific RNA structural motifs [Anal. Biochem. 261 (1998), 183-190]. Its usefulness in principle was demonstrated with binding studies between pyrene-labeled aminoglycosides and 16S ribosomal RNA. The procedure is however unsuitable for the high-throughput screening of large substance libraries, as every single substance to be tested must be provided with a fluorescent label.

The basic object of the present invention is therefore to provide a procedure which makes it possible to characterize interactions between RNA structures and RNA-binding molecules, rapidly, simply and reliably. This procedure should not exhibit the disadvantages of the procedures according to the current state of the art, as shortly described above, and should make it possible to construct a robust, non-radioactive assay which can be automatized, for the identification of substances with potential pharmacological activity which either (a) bind directly to RNA structures or (b) inhibit the interaction between an RNA structure and an associated compound.

This object is solved by providing a procedure with the features characterized in the patent claims. As shown in example 2, the procedure in accordance with the invention may be used particularly for searching for pharmaceutical lead substances to RNA-binding peptides or proteins.

With the procedure in accordance with the invention, substances with specific binding properties for short RNA structural motifs or RNA-binding molecules can be identified and characterized by using reporter ribozymes. The procedure in accordance with the invention exploits the fact that (a) the binding of a molecule to a specific RNA-target motif or (b) the displacement of a molecule from a specific target motif can be directly measured, as the RNA-target motif is structurally linked to a reporter ribozyme domain. An RNA construct of this sort as used in the assay is referred to below as a target reporter construct (TRK). In accordance with the selected embodiment, (a) or (b), two cases can be distinguished:

Case (a): If the RNA-target motif is present in the unbound form, a signal of a certain intensity can be detected. As a consequence of a binding event (e.g. binding of a low molecular weight substance to the target motif), the measured signal is changed. As a result of this, it is possible to detect the binding of the substance to the RNA and to quantify the binding.

Case (b): If the RNA-target motif is present in the bound form (e.g. to an RNA-binding protein) a signal of certain intensity can also be detected. If the RNA-binding molecule is displaced from the binding site on the RNA, the measured signal is changed, making it possible to detect this displacement event. In this way it is therefore possible to identify a substance with the desired binding properties for the original RNA-binding molecule.

The present invention therefore relates to a procedure for the identification of compounds which specifically bind to a desired RNA-target motif and which, as a result, can inhibit or eliminate the function of this motif. This procedure is characterized by the following steps:
(a) Preparing a construct (target-reporter construct; TRK) from a reporter ribozyme domain (I) and the RNA-target motif (II), where (I) and (II) are connected to each other by an RNA-linker and where the reporter ribozyme domain (I) changes its catalytic activity after specific binding of a compound to the RNA-target motif (II);
(b) Producing a signaling riboyzyme substrate which can bind specifically to the reporter ribozyme domain (I) and which can preferentially be cleaved by this;
(c) Bringing into contact the TRK from step (a) and the ribozyme substrate from step (b) with the compound to be identified, for example a candidate from a substance library, or with a mixture containing this substance; and
(d) Determining of the binding of the compound to the RNA-target motif, preferentially as a result of the cleavage of the ribozyme substrate.

On the other hand, the present invention also relates to a procedure for the identification of compounds which can displace a compound which is associated with the desired RNA-target motif (e.g. a compound which is naturally associated with this RNA in the cell) and which can, as a result, inhibit or eliminate the function of this motif. This procedure is characterized by the following steps:
Production of a construct (target reporter construct; TRK) from a reporter ribozyme domain (I) and the RNA target motif (II), where (I) and (II) are connected to each other through an RNA-linker and where the reporter ribozyme domain (I) changes its biological activity after displacement of the compound associated with the desired RNA target motif from the RNA target motif (II);
Production of a signaling ribozyme substrate which can bind specifically to the reporter ribozyme domain (I) and which can, preferably be cleaved by this;
Bringing into contact the TRK from step (a) and the compound associated with the RNA target motif;
Bringing into contact the complex from step (c) and the ribozyme substrate from step (b) with the compound to be identified or with a mixture which contains this compound, for example, using a candidate from a substance library; and
Detection of the displacement of a compound associated with the RNA target motif, preferentially by cleavage of the ribozyme substrate.

The step of the production of the signaling ribozyme substrate, as described above, can be eliminated if a substrate is already known for the reporter ribozyme domain used in the TRK. An embodiment then comprises instead a step for the preparation of a signaling ribozyme substrate and, possibly, the addition of this to the procedure or reaction mixture.

The expression “reporter ribozyme domain”, as used herein, refers to a ribozyme which has been modified so that it is able to, for example, specifically cleave a suitable substrate RNA, giving a measurable signal. Ribozymes, e.g. hammerhead ribozymes (HHR), are catalytic RNA molecules which are capable of cleaving other RNA molecules at phosphodiester bonds in a sequence-specific fashion. The hammerhead ribozyme structure includes three double stranded regions (helices I, II, and III), which flank the cleavable phosphodiester bond, and also two highly conserved single strand sequences [O. Uhlenbeck, Nature 328 (1987) 596-600]. For the purposes of the current invention, all ribozymes are in principle suitable which can cleave phosphodiester bonds in trans, i.e. intermolecularly. Apart from ribonuclease P [C. Guerrier-Takada et al., Cell 44 (1983), 849-857], the known ribozymes which occur in nature (hammerhead ribozyme, hairpin ribozyme, hepatitis delta virus ribozyme, Neurospora mitochondrial VS ribozyme, group I and group II introns) are however self-cleaving or self-splicing catalysts, which act in cis (intramolecularly) [reviewed in P. Turner (eds.), Ribozyme protocols, Humana press (1997), 1-9]. By separating the catalytic unit from the sequence containing the site of cleavage, it was possible in all cases to produce variants of the ribozymes which cleave in trans: hammerhead ribozyme [J. Haselhoff and W. Gerlach. Nature 334 (1988), 585-591]; hairpin ribozyme [A. Hampel and R. Tritz, Biochemistry 28 (1989), 4929-4933]; hepatitis delta ribozyme [M. Been, Trends Biochem. Sci. 19 (1994) 251-256]; Neurospora mitochondrial VS ribozyme [H. Guo et al., J. Mol. Biol. 232 (1993) 351-361]; group I intron from Tetrahymena [Zaug et al., Nature 324 (1986), 429-433]; group II intron [S. Augustin et al., Nature 34 (1990) 383-386]. Separation of the catalytic core sequence from a substrate sequence containing the site of cleavage made it possible to obtain ribozyme variants (corresponding to “reporter ribozyme domains”) which are capable of cleaving almost any target RNA intermolecularly under physiological conditions [J. Haselhoff, W. Gerlach, Nature 334 (1988) 585-591]. The hydrolysis of the target sequence to be cleaved is then always initiated by the formation of a catalytically active complex, consisting of ribozyme and substrate RNA. After cleavage, the hydrolyzed substrate oligonucleotide dissociates from the ribozyme, which is then available for further conversions. Trans-cleaving ribozymes can be developed on the basis of the ribozyme sequence. For that purpose, the ribozyme is subdivided into two regions, where one contains the site of cleavage (substrate) and the other contains the catalytic site (in trans ribozyme). Particularly active in trans ribozymes can be identified by experimental testing, i.e. by the measurement of the cleavage activity of different ribozyme substrate constructs. The synthetic and enzymatic production of ribozymes is known to the expert [Turner (eds.), Ribozyme protocols, Humana press (1997) 51-111]. It has very recently been possible to optimize the kinetic properties (high turnover rates), the sequence length (minimal motifs) and substrate specificity of hammerhead ribozymes. There are, for example, review articles on this subject in Birikh, Eur. J. Biochem. 245 (1997), 1-16; Burke, Nature Biotech. 15 (1997), 414-415 and Eckstein, Lilley (Eds.), Nucleic Acids and Molecular Biology 10, Springer Verlag (1996), 173-329.

The catalytic activity of the ribozyme region then yields a measurable signal in response to the binding of a molecule of the RNA target domain or its displacement, where the term “ribozyme” includes both natural and modified ribozymes and DNA enzymes, the so-called desoxyribozymes (R. Breaker, Chem. Rev. 97 (1997), 371-390; A. Jenne & M. Famulok, Top. Curr. Chem. 202 (1999), 102-131. As an alternative to the ribozymes which cleave nucleic acids, the present invention can also use other suitable signaling ribozymes, for example, ribozymes with RNA-ligase activity. In the case of a ligase ribozyme, the binding event can be detected by PCR [M. P. Robertson & A. D. Ellington, Nat. Biotechnol. 17 (1999), 62-66]. In this case, the detection can preferentially be performed by using the so-called “Taq-man” probes (K. J. Livak et al., PCR Methods Appl. 4 (1995), 357-362].

The term “RNA target motif”, as used herein, relates to RNA molecules or parts of these which fulfill a specific function within the cell on the basis of their sequence or structure. The motifs can then either occur naturally in the cell or be of synthetic nature (e.g. intracellularly expressed RNA aptamers, so-called “intramers”, see following; M. Blind, et al., Proc. Natl. Acad. Sci. USA 96 (1999), 3603-3610).

In the simplest case, the RNA target motif is identical with the reporter ribozyme, i.e. the ribozyme itself is the target. Examples of therapeutically relevant processes which are steered by ribozymes include self-splicing in pathogenic microorganisms, miss-splicing in human cells (e.g. sickle cell anemia), tRNA processing by RNase P or RNA processing of the hepatitis delta virus genome [P. C. Turner (eds.), Methods in Molecular Biology: Ribozyme Protocols, Vol. 74 (1997), Humana Press, Totowa, N. J., USA].

Structurally unique and highly conserved RNA structural elements with an important biological function are an important class of RNA target motifs [A. S. Brodsky & J. R. Williamson, J. Mol. Biol. 267 (1997), 624-639; M. Afshar et al., Curr. Opin. Biotech. 10 (1999), 59-63]. Recognized examples of this include the structural elements “TAR” and “RRE” in the mRNA of the HI virus or the IRES sequence, which is responsible for the specific translation of certain proteins. The significance and strategies for the selection of suitable RNA structural elements are discussed in detail in a recent review article by D. J. Ecker & R. H. Griffey [Drug Disc. Today 4 (1999), 420-429]. The manner in which the structural element was found is nevertheless unimportant for the procedure in accordance with the invention. However, for the signal detection to be as unambiguous as possible, the selected RNA target motif may not be too long, with a preferred maximum length of 60 nucleotides and a more preferred maximum length of 40 nucleotides.

RNA or DNA aptamers are particularly important with respect to the identification of substances directed against RNA-binding proteins [A. D. Ellington & J. Szostak, Nature 346 (1990), 818-822; C. Tuerk & L. Gold, Science 249 (1990), δ 05-510]. Aptamers are artificially selected nucleic acids with specific and sometimes high affinity binding properties to many different molecules [M. Famulok & A. Jenne, Curr. Opin. Chem. Biol. 2 (1998), 320-327; M. Famulok, Curr. Opin. Struc. Biol. 9 (1999), 324-329; S. E. Osborne & A. Ellington, Chem. Rev. 97 (1997), 349-370]. As described in example 2, the claimed procedure can be used to identify lead substances against aptamer-binding molecules. In this context, those aptamers are of particular interest which are directed against disease-relevant (intracellular) proteins (P. D. Good et al., Gene Ther. 4 (1997), 45-54; K. Konopka et al., Drug Target 5 (1998), 247-259; C. Tuerk & S. MacDougal-Waugh, Gene 137 (1993), 33-39; K. B. Jensen et al., Proc. Natl. Acad. Sci. USA 19 (1995), 12220-12224; M. Blind, et al., Proc. Natl. Acad. Sci. USA 96 (1999), 3603-3610).

Aptamers and their intracellular equivalents, the intramers, are target-specific macromolecular lead substances which exhibit important pharmacological properties shared by the later therapeutically active product. Aptamers are therefore extraordinarily well suited for the development of low molecular weight drugs, as the “therapeutic information” stored in the aptamer can be exploited by the procedure in this invention. In principle, a measurable signal is produced in the procedure according to the invention when the macromolecular aptamer is displaced from its binding site on the target protein by another substance. It can therefore be assumed in all probability that the identified substance possesses similar properties as the aptamer (e.g. inhibition of the function of the target protein). In contrast to many other screening assays, the native target protein can be used without potential interference from modifications, such as labeling with dyes or isotopes.

The term “target reporter construct”, as used herein, refers to the coupling of the reporter ribozyme domain and the RNA target motif through an RNA linker (see definition below), where the coupling occurs in such a way that the reporter ribozyme domain changes, keeps or loses its biologically active conformation after specific binding of a specific ligand (the compound to be identified) to the RNA target motif. The expert can produce suitable target reporter constructs with the help of techniques which have now been established (Soukup and Breaker, Current Opinions in Structural Biology 10 (2000), 318-325). The present definition also includes TRKs in which the reporter ribozyme domain and the RNA target motif are identical, and the presence of an RNA linker is eliminated, especially in this case.

In the context of the target reporter construct (TRK), the aptazyme (aptamer-ribozyme) will be discussed here shortly. RNA molecules the function of which can be regulated by protein binding play an important role in many cellular processes [K. J. Addess et al., J. Mol. Biol. 274 (1997), 72-83; M. J. Gait & J. Karn, Trends Biochem. Sci. 18 (1993), 255-259]. It has recently been shown that controllable RNAs—in this case allosteric ribozymes—can be obtained by rational design or in vitro selection. This was based on the fact that the spatial structure of a ribozyme can be stabilized or destabilized by structural changes and that major structural changes affect the catalytic activity of the ribozyme in most cases.

In addition, the fact was exploited that structural changes in RNA molecules can be coupled to the binding of a ligand. Breaker et al. succeeded in producing structural changes in hammerhead enzymes by evolutive methods or by rational design in such a way that the catalytic activity of the ribozymes could be structurally regulated by binding a low molecular weight ligand [J. Tang & R. R. Breaker, Chem. Biol. 4 (1997), 453-459; J. Tang & R. R. Breaker, RNA 3 (1997), 914-925; J. Tang & R. R. Breaker, NAR 26 (1998), 4214-4221; G. A. Soukup & R. R. Breaker, PNAS 96 (1999), 3584-3589]. Ellington et al. applied the principle of allosteric regulation to ligase ribozymes [M. P. Robertson & A. D. Ellington, Nat. Biotechnol. 17 (1999), 62-66].

The term “aptazyme” has now become established in the literature for these ribozymes which can be allosterically regulated. An aptazyme is characterized by two structural domains which are independent of each other—a catalytically active RNA motif (“ribozyme”) and a ligand-binding RNA-motif (“aptamer”), which serves as a receptor domain. Binding of a ligand to the receptor domain causes structural changes, which result in increases or decreases in the catalytic activity. Aptazymes are therefore suitable, for example, as molecular switches for the detection of low molecular weight substances or as control units for the conditional control of gene expression [see e.g. WO 94/13791 and PCT 98/08974]. This also applies to the oligonucleotides in accordance with the invention.

In the target reporter construct in accordance with the invention, the RNA target motif must be linked to the ribozyme domain in a defined manner. The target ribozyme construct is based on the previously described general principle of allosterically controllable ribozymes and must in principle exhibit the following properties.

The term “RNA linker”, as used herein, relates to an RNA sequence which connects the reporter ribozyme domain and the RNA target motif in such a way that signaling is communicated by conformational changes in the RNA structure. This “RNA connecting link” is of particular importance in the procedure according to the invention [G. A. Soukup & R. R. Breaker, Structure 7 (1999), 783-791; G. A. Soukup & R. R. Breaker, Trends Biotech. 17 (1999), 469-476]. Suitable connecting links can be selected either by empirical testing of different known sequences or by in vitro selection [G. A. Soukup & R. R. Breaker, PNAS USA 96 (1999) 3584-3589].

The term “signaling ribozyme substrate”, as used herein, relates to any RNA molecule which can bind specifically to the reporter ribozyme domain, which is cleaved by it when it is in its biologically active conformation and which permits detection of this cleavage. This assumes that the cleaved ribozyme substrate can be distinguished from the uncleaved ribozyme substrate and that a directly detectable signal is produced.

For example, the ribozyme substrate may have an anchor group at one end, which allows its immobilization to a suitable matrix, and has at the other end a reporter group, which serves to detect the immobilized (uncleaved) ribozyme substrate. When the TRK is inactive (i.e. when there is no suitable ligand for the RNA target motif), the ribozyme substrate remains intact and can be simply detected after immobilization on the matrix, as the anchor group is still bound to the reporter group. In contrast, when the TRK is active (i.e. after binding of the ligand to be identified to the RNA target motif), the reporter-specific signal is not detectable, as the reporter group is separated from the anchor group, as a result of the cleavage of the substrate. As an alternative to an anchor group (such as biotin), the ribozyme substrate can also be immobilized by complementary sequence hybridization, if both, cleavage site and reporter group are distal to the hybridization site. Reporter groups which are simple to detect and which are easy to couple to the ends of nucleic acids include ³²P, dye molecules and molecules which are detectable with labeled antibodies. However, it is also possible to detect the cleaved ribozyme substrate by a series of other procedures which are known to the expert, comprising gel electrophoresis and PCR.

The signaling ribozyme substrate is essentially complementary to the sequence or sequences of the reporter ribozyme domain which are responsible for substrate binding, i.e. the substrate exhibits complementarity which allows binding to the ribozyme in such a manner that effective and specific cleavage of the ribozyme substrate is guaranteed. Preferably, the ribozyme substrate is fully complementary to the sequences of the reporter ribozyme domain which are responsible for substrate binding. The length of the binding region of the ribozyme substrate is preferably 8 to 14 nucleotides [P. Turner eds., Ribozyme protocols, Humana press (1997), 151-159, 253-264]. The ribozyme substrate may contain additional sequences at its 5′- and/or 3′-end which do not participate in the binding to the ribozyme.

The compounds to be identified or candidate compounds can in principle be any compounds of the widest variety of types. The expert is also aware of a variety of sources which contain compounds which are suitable for the screening procedure in accordance with the invention. For example, all conceivable substance libraries are suitable, including antisense nucleic acids; however, libraries of low molecular weight molecules are preferred, which fulfill certain conditions, for example, with respect to low toxicity [D. J. Ecker & R. H. Griffey, Drug Disc. Today 4 (1999), 420-429].

The constructs which are necessary for the procedure in accordance with the invention (TRK, ribozyme substrate) and the oligonucleotides in accordance with the invention are preferably prepared in larger quantities by in vitro transcription of the corresponding DNA sequences. For this purpose, DNA sequences are inserted into a vector which allows replication of the inserted DNA in a suitable host, under the control of a suitable promoter, preferably the T7 promotor. Suitable vectors for the replication of prokaryotic or eukaryotic systems include, for example, pBR322, pNEB193, pUC18, pUC19 (Biolabs, USA.) [J. Sampson and O. Uhlenbeck, Proc. Natl. Acad. Sci. USA 85 (1988), 1033-1037]. The plasmids are subsequently isolated, purified and the in vitro translation is performed in accordance with standard procedures. The constructs used for the procedure in accordance with the invention can however also be prepared by automated solid phase synthesis in accordance with standard procedures.

In a preferred embodiment of the procedure in accordance with the invention, the reporter ribozyme domain is derived from a hammerhead ribozyme. Reference is made to the above discussion on hammerhead ribozymes and the production of variants which perform intermolecular cleavage.

In a preferred embodiment, the above or signaling ribozyme substrate is doubly labeled and the cleaved substrate is easy to distinguish from the intact substrate. For example, a terminally biotinylated ribozyme substrate can be used, which is labeled with fluorescein at its other end. After the reaction has been completed, the sample is incubated with a streptavidin-coated solid phase (e.g. with a commercially available microtiter plate), to permit coupling of the biotinylated substrate end to the streptavidin matrix. The matrix is then washed and tested. If a ligand is present which binds specifically to the RNA target motif (reporter ribozyme domain is activated), there is no measurable fluorescein-specific fluorescence, or only weak and non-specific background fluorescence, as the fluorescein-labeled cleavage product could not be immobilized. If however there is no activation of the reporter ribozyme construct, because of the absence of a ligand which binds specifically to the RNA target motif, the proportion of uncleaved and immobilized ribozyme substrate can be quantified by measurement of the fluorescein-specific fluorescence.

In a particularly preferred embodiment of the procedure in accordance with the invention, the doubly labeled ribozyme substrate contains a fluorophore group and a group which quenches fluorescence, whereby, after cleavage by the reporter ribozyme domain, the quenching of the fluorescence of the fluorophore by the quenching group is prevented. Ribozyme substrates labeled in this way can, for example, be used in the FRET procedure [FRET=Fluorescence resonance energy transfer (J. R. Lakowicz, Principles of Fluorescent Spectroscopy; Plenum Press, New York (1983)]. FRET oligonucleotides are, for example, described in K. J. Livak, S. J. A. Flood, J. Marmaro, W. Giusti, K. Deetz, PCR Meth. Appln. 4 (1995), 357-362.

Particularly preferred FRET ribozyme substrates are RNA oligonucleotides or DNA-RNA hybrids in which a fluorescent group (e.g. FAM=6-carboxyfluorescein, TET=tetrachloro-6-carboxyfluorescein or HEX=hexachloro-6-carboxyfluorescein) and a corresponding fluorescence quenching group, a so-called “quencher”, (e.g. sulforhodamine 101, TAMRA=6-carboxytetramethylrhodamine or Cy 3), are incorporated close enough to each other for there to be effective quenching of the fluorescence of the fluorophore [Lakowicz, Principles of Fluorescence Spectroscopy, Plenum Press, New York (1983), 303-339; V. Förster, Annals of Physics (Leipzig) 2 (1948), 55-75]. After cleavage of the ribozyme substrate by ribozyme-catalysed hydrolysis of a specific phosphodiester bond, the cleavage products can separate from each other in solution: the fluorescence of the fluorophore is now no longer subject to intramolecular quenching. If therefore a suitable ligand binds to the RNA target motif, leading to a biologically active reporter ribozyme domain, this can be determined by production of a measurable fluorescence signal by cleavage of the substrate. The procedure in accordance with the invention based on FRET technology is particularly suitable for the industrial high-throughput screening of substance libraries, as it is simple to perform and easy to adapt to different formats of microtiter plates [X. Chen et al., Genome Res. 8 (1998), 549-556; K. P. Bjornson et al., Biochemistry 33 (1994), 14306-14316; A. R. Gelsthorpe et al., Tissue Antigens 54 (1999), 603-614; J. E. Gonzalez et al., Drug Discov. Today 4 (1999), 431-439]. In particular, radioactive waste is avoided, which otherwise must be expensively disposed of. Moreover, this embodiment of the procedure in accordance with the invention has the advantage that it very sensitively detects the binding of the ligand, as the catalytic cleavage of the FRET substrate leads to clear signal amplification [for the very rapid determination of the cleavage activity of hammerhead enzymes by fluorescence measurement in the microtiter plate format, see also Jenne et al., Angew. Chem. 111 (1999), 1383-1386.]. The embodiment of the procedure in accordance with the invention based on FRET technology is preferably designed so that initially, before the binding or displacement event which is to be measured, no or only a very small signal is measured and there is only a clear change in the fluorescence signal as a consequence of the binding or displacement. This is mainly achieved as the result of selection of a suitable RNA linker, which connects the RNA target sequence to the ribozyme domain (see above). Methods to label ribonucleic acids with fluorophores or with fluorescence-quenching groups and techniques to measure energy transfer (quenching) have already been described in detail [Turner (ed.), Ribozyme protocols, Humana press (1997), 241-251]. 5′-Fluorophore- and 3′-quencher-labeled RNA-oligonucleotides are commercially available (e.g. 5′-FAM- and 3′-TAMRA-labeled RNA from Eurogentec, Belgium). It is favorable to perform the labeling at the RNA ends, so as not to influence the hybridization in the reporter ribozyme domain.

To avoid fluorescence emission from unwanted cleavage (e.g. by nucleases) it is particularly advantageous to use nuclease-resistant ribozyme substrates (Eaton and Pieken, Annu. Rev. Biochem. 64 (1995), 837-863 and Shimayama et al., Nucleic Acids Res. 21 (1993), 2605-2611). This is especially advantageous with respect to in vivo applications, in which the doubly labeled substrate is transfected exogenously into cells by suitable techniques (e.g. microinjection, liposome transport, etc.) (P. Turner (ed.), Ribozyme protocols, Humana press (1997), 417-451). In a particularly preferred embodiment, the double labeled substrates are then modified RNA oligonucleotides. As long as the site of cleavage in the substrate is NUH ↓, (according to the IUB Code.: N=any base, H=A, U or C), the substrate can contain desoxyribonucleotides and/or modified bases and/or 2′-modified ribose units. This increases the stability of the substrate in the cell extract (N. Taylor et al., Nucleic Acids Res. 20 (1992), 4559-4565). The use of internally labeled rather than end labeled substrates can also contribute to an improvement in the signal to noise ratio, as fluorescence quenching may be enhanced when the distance between the two groups (fluorophore and quencher) is shorter.

The present invention also relates to a drug which contains a compound which has been identified by a procedure in accordance with the invention. This also includes a compound derived from this, which can also bind to the RNA target motif, whereby this compound, for example, only includes a portion or a partial sequence of the originally identified compound or a portion or partial sequence which differs from this, with differing, but preferably enhanced affinity to the RNA target motif, in comparison to the original compound. The drug is preferably combined with a suitable carrier. Suitable carriers and the formulation of drugs of this sort are familiar to the expert. For example, suitable carriers include phosphate-buffered saline, water, emulsions, for example, oil/water emulsions, wetting agents, sterile solutions, etc. The administration of the drug can be either oral or parenteral. The procedures for parenteral administration include topical, intra-arterial, intramuscular, subcutaneous, intramedullar, intrathecal, intraventricular, intravenous, intraperitoneal or intranasal administration.

Finally, the present invention relates to a kit (or assay) for the performance of the procedures in accordance with the invention, where the kit includes the following compounds, as described above: (a) a target reporter construct (TRK) and (b) a signaling ribozyme substrate.

In a further aspect the invention relates to a polynucleotide comprising a hammerhead ribozyme and an aptamer which is specific for a target molecule, the aptamer portion of the polynucleotide preventing the formation of the catalytically active ribozyme by base pairing with sequences of the hammerhead ribozyme. This is achieved in that the sequence which is essential for the catalytic activity of the hammerhead ribozyme, or a part of this, is, as a result of base pairing with the aptamer sequence, no longer available for the base pairing which is required for the catalytic activity.

A further aspect of the invention relates to a polynucleotide comprising a hammerhead ribozyme and an aptamer, the aptamer being specific for a target molecule, in particular a polynucleotide in accordance with the above aspect of the invention, which also includes the bound target molecule which is specific for the aptamer, and where the binding of the target molecule to the aptamer sequence leads to the development of enyzmic activity in the ribozyme.

Yet another aspect of the invention relates to a polynucleotide comprising a hammerhead ribozyme and an aptamer for a target molecule, where the binding site of the catalytic domain of the ribozyme for a ribozyme substrate is blocked in the absence of the target molecule of the aptamer for binding of a substrate of the ribozyme.

A further aspect of the invention relates to a polynucleotide comprising a hammerhead ribozyme and an aptamer for a target molecule, in particular one in accordance with one of the other aspects of the present invention, where the binding site of the catalytic domain of the ribozyme is accessible for a ribozyme substrate in the presence of the target molecule of the aptamer for binding of a substrate of the ribozyme.

Finally, another aspect of the invention relates to a polynucleotide comprising a hammerhead ribozyme and an aptamer for a target molecule, in particular a polynucleotide in accordance with one of the other aspects of the present invention, where the pattern of base pairing of the polynucleotide on binding of the target molecule to the aptamer is different from that of the polynucleotide in the absence of the target molecule of the aptamer, in particular, when the target molecule does not bind to the aptamer.

In other words, the present invention also relates to a polynucleotide which comprises a hammerhead ribozyme and an aptamer which is specific for a target molecule, where both, the hammerhead ribozyme and the aptamer, are joined to each other and give different base pair hybridization patterns in the ribozyme, depending on the presence of the target molecule which is specific for the aptamer, which has the following effects: Binding of the target molecule to the aptamer leads to the formation of a catalytically active ribozyme in the polynucleotide in accordance with the invention. In contrast, if the target molecule does not bind to the aptamer, the catalytically active ribozyme in the polynucleotide in accordance with the invention is not formed.

For example, the target molecule cannot bind to the aptamer sequence when a binding partner of the target molecule, for example, an inhibitor is present, which prevents a specific interaction between the aptamer nucleic acid part of the polynucleotide in accordance with the invention and the target molecule.

The polynucleotides described above are therefore both states of a polynucleotide, which are defined by the binding or non-binding of the target molecule of the aptamer. The two states differ not only in their different secondary and tertiary structures, but additionally and particularly by the type and, in some cases, numbers of base pairs formed in the polynucleotide. In other words, the two states differ in their patterns of base pairing. This change in base pairing pattern constitutes a difference between the polynucleotides in accordance with the invention and the ribozymes known in the state of the art, including the allosteric ribozymes and the aptazymes, in which the allosteric effect of the binding of the allosteric effectors, such as the target molecule of the aptamer, is only evident as a change in the secondary and/or tertiary structure, but not in a change in the pattern of base pairing.

In the sense of the present disclosure, the oligonucleotides in accordance with the invention correspond to target receptor constructs, the hammerhead ribozymes to receptor ribozyme domains and the aptamers to RNA target motifs. In the context of the present invention, the aptamer may also consist of deoxyribonucleic acid.

The herin described hammerhead ribozymes are typically those which consist of three helices, two of which form their “target RNA” by hybridization with the complementary sequence and the third of which is formed by a double strand within the ribozyme. The catalytic domain of the ribozyme consists of nucleotides which are arranged between the double stranded structures. The term “target RNA” means the RNA substrates which can be cleaved in cis or in trans by hammerhead ribozymes. In the context of the present invention, the target RNA is typically RNA which is a substrate for the corresponding ribozyme, particularly a signaling substrate, in the sense of the present invention. In one embodiment of the polynucleotides in accordance with the invention, it is planned that these include a substrate for the catalytic activity of the ribozyme. Substrates of this type have already been described in the context of the above aspects of the invention, in particular the elaboration of the substrate as a FRET substrate. Reference is made here to the corresponding disclosure.

In a further preferred embodiment, the polynucleotide in accordance with the invention exhibits a sequence which is selected from the group comprising SEQ ID No. 51 and SEQ ID No. 52. The polynucleotide in accordance with the invention can preferably embody RNA. In the context of the present invention, at least regions of the polynucleotide in accordance with the invention may also consist of DNA, particularly those regions which participate in pairing with the substrate and also those which participate in intramolecular base pairing. An example of substrates for the polynucleotides in accordance with the invention is the nucleic acid disclosed in SEQ ID No. 53.

The configuration underlying the polynucleotides in accordance with the invention gives surprising advantages in comparison with the allosteric ribozymes known in the state of the art. The most important of these advantages should be seen in the fact that the polynucleotides in accordance with the invention make it possible to produce an allosteric ribozyme for which the allosteric effector, i.e. the target molecule which binds to the aptamer part of the polynucleotide, can be almost freely selected, without there being resulting impairment of the functionality of the allosteric ribozyme. This is a difference between the polynucleotide in accordance with the invention and the aptazymes described according to the state of the art, in which a so-called communication module is interposed between the receptor part or domain, i.e. the part of the allosteric ribozyme which binds the target molecule, and the catalytically active ribozyme domain, which is referred to here as the RNA linker. These communication domains have a really considerable influence on both the effectiveness and the possibilities for designing the allosteric ribozyme, in particular concerning the choice of the aptamer to be used. In some cases, this has the consequence that allosteric ribozyme systems of this sort cannot be used for certain target molecules and then, for example, cannot be used in the context of screening of compounds such as candidate compounds, which influence the interaction between the aptamer and the target molecule. The procedures described here, using the polynucleotides in accordance with the invention, are practically free of this type of restriction. The polynucleotides in accordance with the invention are namely standardized or easily standardizable allosteric ribozymes or target receptor constructs, of which the specificity can be surprisingly easily adjusted to each molecule to which an aptamer can be produced. The reason for the flexibility of the specificity of the oligonucleotides in accordance with the invention, i.e. the allosteric ribozymes in accordance with the invention, is based on the structure of the hammerhead ribozymes. As three helices must be formed, it is possible to design at least one helix in such a way that it is complementary to the nucleic acid sequence of the aptamer part of the allosteric ribozyme. As these double stranded regions of the hammerhead ribozymes have no catalytic function, any aptamer at all can be introduced into the allosteric ribozyme, with the result that, when the target molecule of the aptamer binds to the aptamer part of the allosteric ribozyme, the helices or non-helical nucleotides which are required for the catalytic activity of the ribozyme are present as such and are not paired with at least a part of the aptamer sequence. The allosteric ribozyme is only catalytically active in the form that binds target molecule.

It follows from the above that binding of the target molecule to the aptamer part of the allosteric ribozyme results in another base pairing or base hybridization pattern in the oligonucleotide, than that in the absence of the target molecule. It is this change in the pattern of base pairing and the resulting accessibility or non-accessibility of the catalytic part of the hammerhead ribozyme which differentiates the polynucleotide in accordance with the invention from the state of the art and which is the reason for the surprising advantage of the oligonucleotides in accordance with the invention.

A further aspect of the present invention relates to a biosensor comprising a polynucleotide in accordance with the invention, whereby it is planned in a preferred embodiment that the polynucleotide is bound to a solid carrier. The basic possibilities for the use of biosensors also apply to the embodiments in accordance with the invention.

In yet another aspect, the invention relates to a procedure for the identification of a compound which binds to a target molecule and which includes the following steps:
a) Providing a polynucleotide in accordance with one of the claims 10 to 16,
b) Optionally determining the catalytic activity of the ribozyme,
c) Adding the target molecule, the target molecule interacting with the nucleic acid which binds the target molecule,
d) Optionally determining the catalytic activity of the ribozyme,
e) Adding a candidate compound,
f) Optionally determining the catalytic activity of the ribozyme,
g) Providing a substrate for the catalytic activity of the polynucleotide and adding the substrate to the reaction mixture,
h) Measuring the binding of the candidate compound to the target molecule.

The term candidate compound is used here to designate a compound which is used in these procedures and is investigated to establish whether it binds to the target molecule and, in particular, at a site which is recognized by the aptamer which is specific for the target molecule. Candidate molecules of this sort are in principle suitable as pharmaceutical lead substances, provided that they influence the interaction between the aptamer and the target molecule. In particular, a candidate compound is also a “compound to be identified” in the sense of the present disclosure.

It is planned in a preferred embodiment that the candidate compound should initially be tested for its ability to influence the interaction between the aptamer and the target molecule. This can, for example, happen in that an allosteric ribozyme in accordance with the invention or an oligonucleotide in accordance with the invention are used which comprises an aptamer, which binds to the target molecule, but possibly at another site, and which then allows a limitation of the site at which there is an interaction between the aptamer and the target molecule. The allosteric ribozymes used herein, more exactly aptazymes, differ in their specificity and/or affinity to the corresponding target molecules or parts of these. It is also included in the context of the present invention that, after the candidate compound has been identified or validated by the procedure in accordance with the invention, it is subjected to a further identification or validation step, where the above applies and an appropriately planned procedure in accordance with the invention is used. In the context of this additional, prior or subsequent, validation step, a procedure can also be used as shown, for example, in FIG. 8a. An allosteric ribozyme is then provided which includes an aptamer which is specific for the same target molecule, as used in the context of the procedure in accordance with the invention, in which there is a change in the pattern of base pairing as a result of binding of the target molecule. The catalytic activity of the ribozyme part of the allosteric ribozyme is optionally determined in advance and then the target molecule is added to the reaction mixture in accordance with the invention, whereby the target molecule interacts with the aptamer domain in the polynucleotide which binds the target molecule. The catalytic activity of the allosteric enzyme in the presence of the target molecule can then, again, optionally be determined and then the candidate compound is added. There is a further optional determination of the catalytic activity of the allosteric ribozyme in the presence of target molecule, before the candidate compound is added. Followed by another optional determination of the catalytic activity of the allosteric ribozyme in presence of target molecule and candidate compound, the substrate for the catalytic activity of the polynucleotide is finally provided and added to the reaction mixture of the components named above. Finally, the binding of the candidate molecule to the target molecule is determined with the help of the procedure in accordance with the invention. In one embodiment, the allosteric ribozyme includes a hammerhead ribozyme part and an aptamer part, possibly joined together by a linker, for example an RNA linker, as described here, where the aptamer is directed against a molecule of molecular weight above 300 Da, preferably above 1000 Da, more preferably above 2 kDa and even more preferably of more than 5 kDa. In preferred embodiments, the target molecule is a peptide or protein. A protein-binding allosteric ribozyme of this type in accordance with the invention is described in the examples.

With the procedures in accordance with the invention, in particular with those which use allosteric ribozymes which bind to peptide or protein target molecules, it has been possible for the first time to use an inhibition assay to also identify those compounds which interact with peptides or proteins. As peptides and proteins participate in many biological and pathological processes, the provision of allosteric ribozymes in accordance with the invention and their use in a procedure in accordance with the invention makes it possible for the first time to determine lead structures which bind to this highly relevant group of target molecules and which can therefore be used, either directly or indirectly, in the development of pharmaceutically active substances.

In a preferred embodiment, it is planned to couple the two procedures shown in FIG. 8 for the identification of compounds which specifically interact with a target molecule, i.e. the compound which has already been identified or validated in the procedure shown in FIG. 8a will be additionally identified or validated in the procedures shown in FIG. 8b. The advantage of coupling the two procedures is that the inherent disadvantages of the two procedures with respect to false positive or false negative results can compensate each other. In particular, false positive results can arise if the candidate compound directly affects the catalytic activity of the ribozyme, for example by binding to the ribozyme part of the aptazyme, and then the effect observed in the experiment is not due to an interaction between the candidate compound and the target molecule. Especially, since in one of the two procedures the polynucleotide in accordance with the invention is used in which binding of the target molecule causes a change in the base pair hybridization pattern and the resulting easy adaptability to different target molecules accompanying this kind of polynucleotides, coupling of the two procedures leads to particularly reliable identification of compounds which react specifically with the target molecule.

In particular, it is intended that, before step f) or after step g) of the procedure in accordance with the invention, the candidate compound should undergo a procedure which includes the steps:
a) Providing a polynucleotide, whereby the polynucleotide is an allosteric ribozyme and comprises a hammerhead ribozyme and an aptamer which is specific for the target molecule, preferably the aptamer of the polynucleotide in accordance with claim 19,
b) Optionally determining the catalytic activity of the ribozyme,
c) Adding the target molecule, whereupon the target molecule interacts with the nucleic acid which binds the target molecule,
d) Optionally determining the catalytic activity of the ribozyme,
e) Adding a candidate compound,
f) Optionally determining the catalytic activity of the ribozyme,
g) Providing a substrate for the catalytic activity of the polynucleotide and adding the substrate to the polynucleotide, and
h) Determining the binding of the candidate molecule to the target molecule.

In the procedure in accordance with the invention, it is intended that the binding of the candidate compounds to the target molecule should be determined by measuring the catalytic activity of the ribozyme. The catalytic activity of the ribozyme is determined by using the FRET substrate, as herein initially described.

It is intended in a further embodiment that the substrate should contain a fluorophore and a fluorescence quencher group, where the quenching of the fluorescence is removed, or at least reduced, after cleavage of the substrate by the catalytic activity of the ribozyme or the catalytic domain of the polynucleotide in accordance with the invention. In a particularly preferred embodiment, it is intended that the fluorophore group is 6-carboxyfluorescin and the fluorescence quencher group is 6-carboxytetramethylrhodamine or “Cy₃”.

A further aspect of the present invention relates to a drug which includes a compound which has been identified by a procedure in accordance with the invention.

Yet another aspect of the invention relates to a kit for the performance of the procedure in accordance with the invention and which comprises:
a) a polynucleotide in accordance with the invention and
b) a signaling ribozyme substrate.

Yet another aspect of the present invention relates to the use of coumermycin for the production of a drug to treat HIV and/or FIV.

Another aspect of the invention relates to the use of nosiheptide for the production of a drug for the treatment of HIV and/or FIV.

Yet another aspect of the invention relates to the use of patulin for the production of a drug for the treatment of HIV and/or FIV.

Another aspect of the present invention relates to the use of coumermycin, nosiheptide or patulin for the treatment of HIV and/or FIV.

Another aspect of the invention relates to the use of the compounds which were identified or validated in accordance with the invention, e.g. coumermycin, nosiheptide or patulin, for the development of pharmaceutically active compounds which can eventually be used as drugs. The procedure can be as follows. Firstly, it is possible that derivatives of the identified compounds can be prepared the pharmacological properties of which are optimized. For example, such properties are efficacy, side-effects, toxicity, etc. On the other hand, it is possible that, on the basis of the identified compounds, those elements, groupings or structures can be identified which a pharmaceutically active compound necessarily exhibits. In particular, a corresponding profile can be developed on the basis of several compounds once these have been identified.

It lies in the context of the present invention that the ribozyme substrate for the catalytic activity of the ribozyme is covalently bound to the ribozyme or to the polynucleotide which comprises the aptamer, in accordance with one of the aspects of the present invention, so that the catalytic activity of the ribozyme is an intramolecular reaction. The binding between the ribozyme and the ribozyme substrate is typically an internucleoside bond, which can also be present at both ends of the ribozyme or ribozyme substrate, or within the ribozyme or the nucleic acid sequence corresponding to the ribozyme substrate. If the ribozyme is elaborated in this way, the steps in the procedure in accordance with the invention are simplified, so that separate provision of the ribozyme substrate can be dispensed with, although the other steps which characterize the procedure in accordance with the invention are retained.

The figures and examples below are used to further explain the invention in more detail, and give rise to further characteristics, embodiments and advantages.

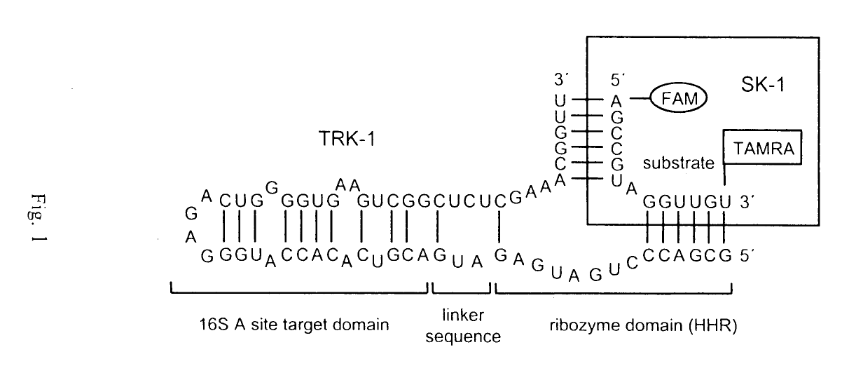

FIG. 1: Secondary structures of construct TRK1 and substrate SK1.

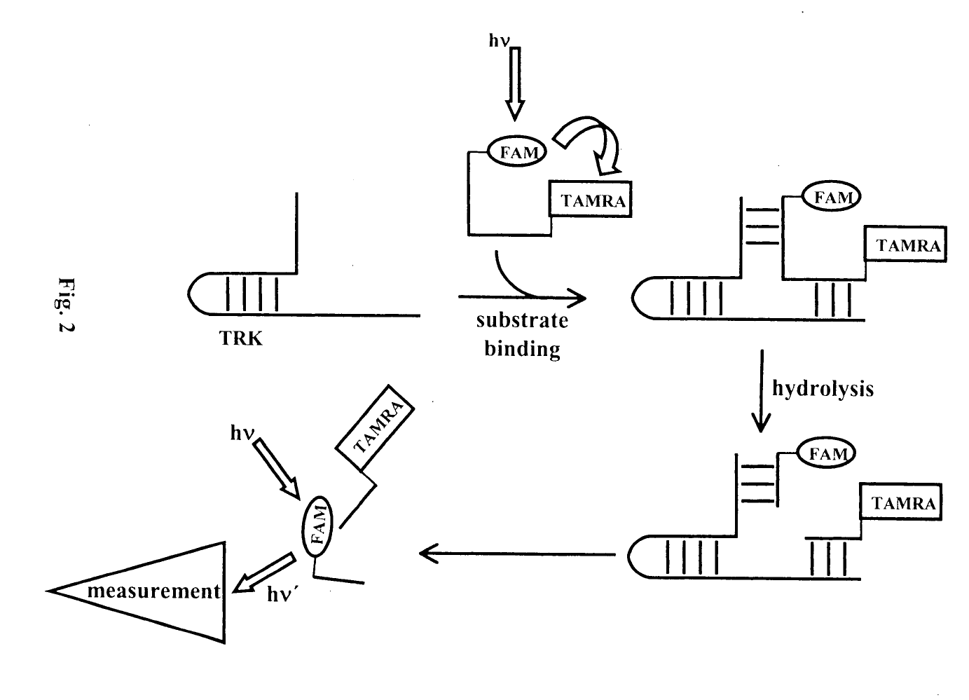

FIG. 2: Principle of the fluorescence measurement: A so-called FRET oligonucleotide (SK1) was selected as substrate for the reporter ribozyme TRK-1. This specific substrate includes a fluorophore group (FAM=6-carboxyfluorescein) which is spatially close to a molecule which quenches fluorescence (TAMRA=6-carboxytetramethyl rhodamine), so that the fluorescence emission of the fluorophore group is effectively quenched. The binding of the doubly labeled FRET substrate leads to the formation of the catalytic complex. After hydrolysis of the FRET substrate by the ribozyme, the cleavage fragments can be separated from each other in solution. The fluorescence of the fluorophore is then no longer intramolecularly quenched and the ribozyme is available for the next catalytic cycle. The removal of the FRET effect leads to a detectable increase in the FAM-specific fluorescence in the sample. As the increase in fluorescence is directly proportional to the cleavage activity of the ribozyme, the rate of the reaction can be determined from the measured data.

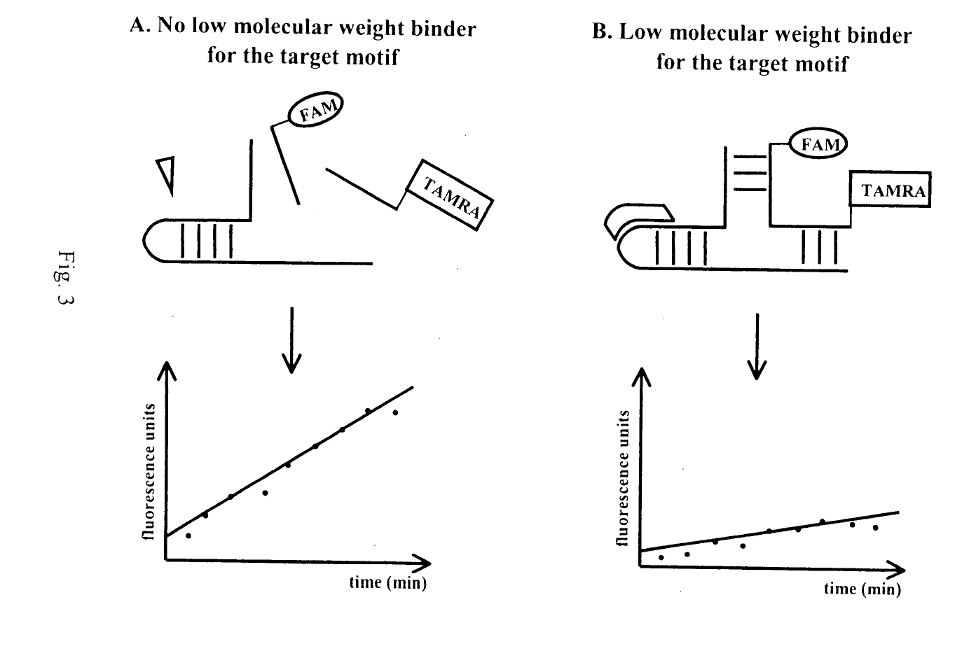

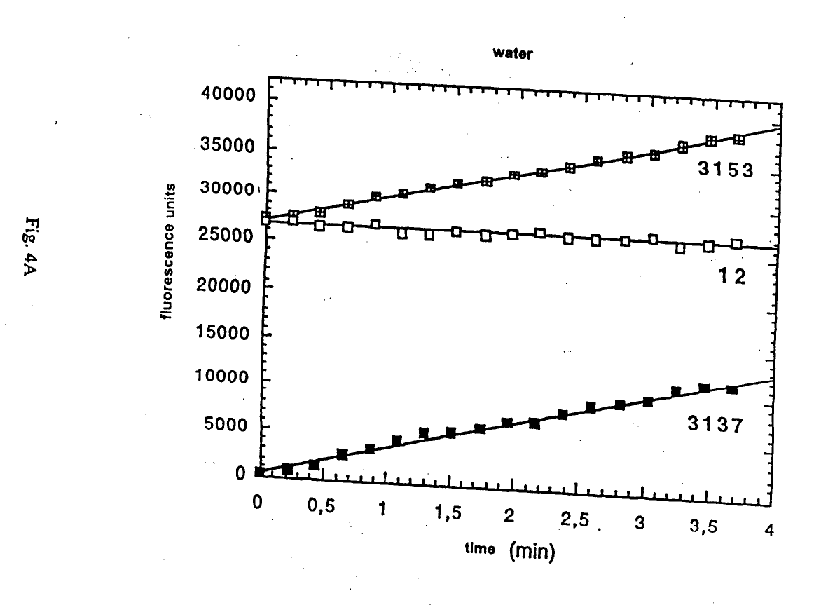

FIG. 3: Principle of the identification of RNA-binding substances from combinatorial compound libraries. The case is presented that the binding of a substance leads to a reduction in the fluorescence measured. The results are evaluated by plotting the measured fluorescence against the initial reaction rate. Comparison of the rates of reaction (=slope of the curve) is used to detect the interaction between RNA-binder and RNA. In the illustrated example, the slope for (B) is significantly lower than for (A).

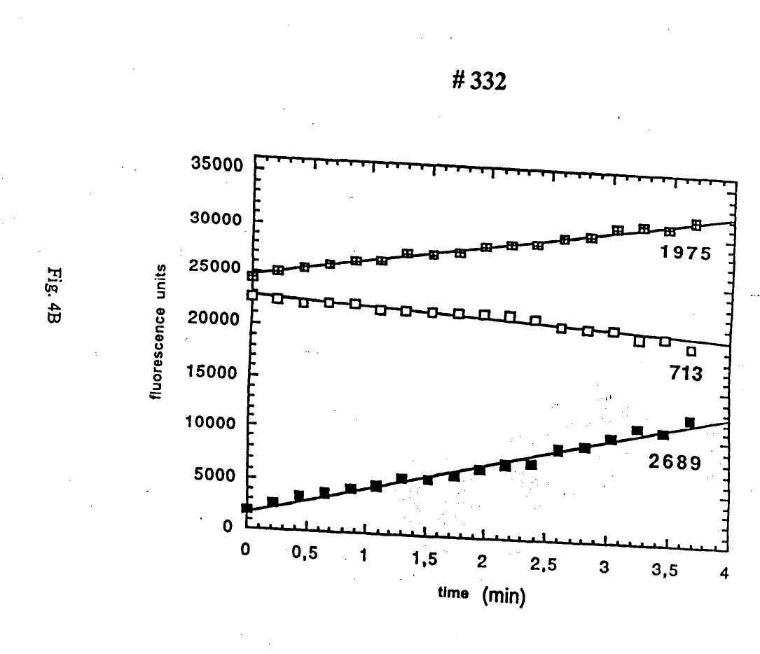

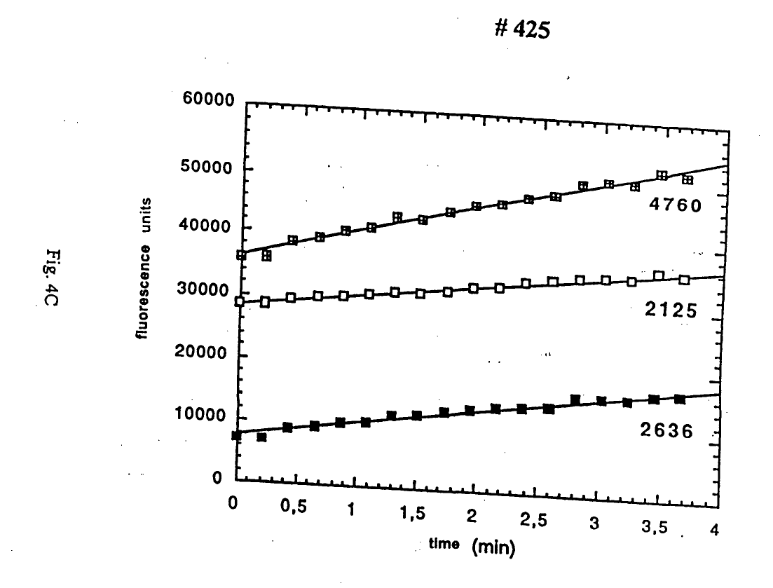

FIG. 4: Fluorescence measurements with TRK1/SL1 which were obtained by screening the substance library. Time-dependent increase of the fluorescence signal for (a) the non-inhibited TRK1 reaction, (b) the TRK1 reaction in the presence of substance #332 and (c) the TRK1 reaction in the presence of substance #425. For each, the measured values, the regression lines for the uncorrected course of the reaction, the corresponding control reaction mixture without TRK1 and the corrected reaction are shown. The values for the initial rates of the reaction (=slope of the corresponding regression lines) are also given.

FIG. 5: Principle of the screening assay. Each microtiter plate contains (i) the target protein to be examined, (ii) a substance from the compound library (iii) and a suitable reporter ribozyme construct, for example, an intramer-ribozyme construct (see reporting text). Fluorescence measurements can be used to identify substances which are capable of binding to the target protein being examined. As this results in displacement of the RNA ribozyme construct from the protein binding domain, there is a detectable change in the fluorescence emission. The fluorescence signal is enhanced in the example illustrated. Microtiter wells A and C in the illustration contain no target protein-binding substance, so that only a weak signal is detected. In contrast, well B gives a rapidly increasing signal, as this well contains a substance with the desired target-specific properties.

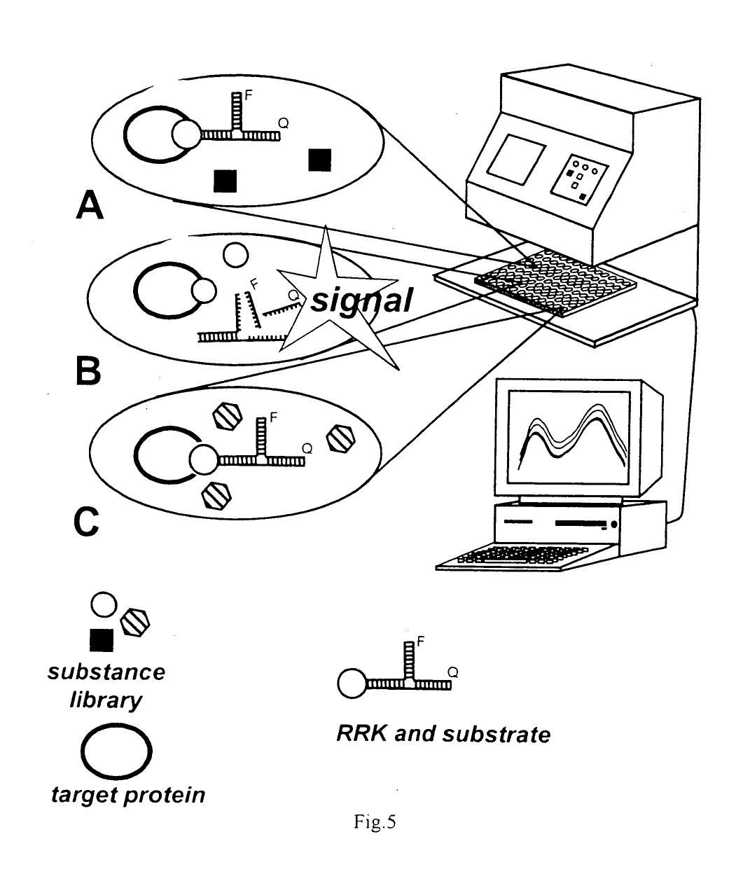

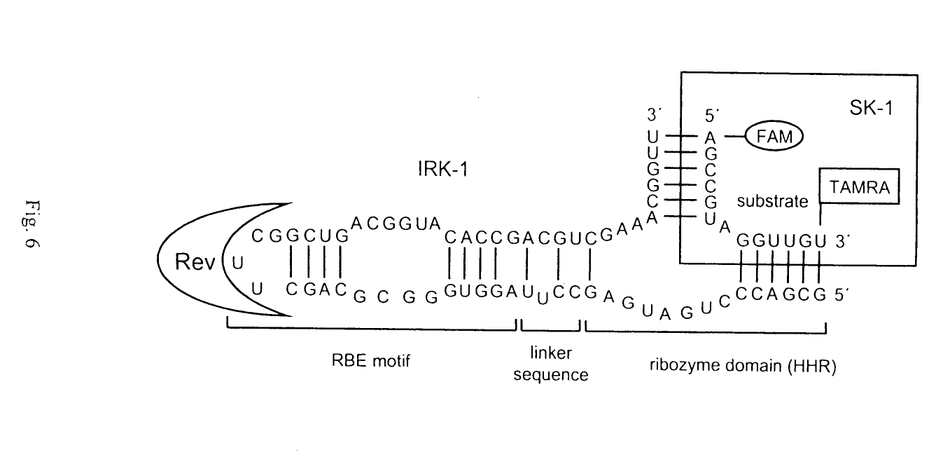

FIG. 6: Secondary structures of reporter ribozyme construct IRK1 and ribozyme substrate SK1. The selected Rev-binding sequence was wt RBE motiv, as isolated by D. P. Bartel et al. [Cell 67 (1991), 529-536].

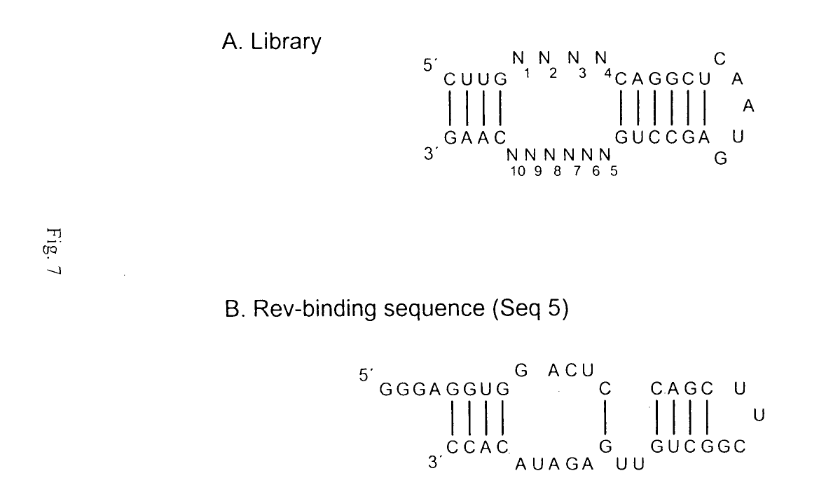

FIG. 7: Secondary structures of library (A) and of the isolated Rev-binding sequence (Seq 5) (B) [L. Giver et al., NAR 23 (1993), 5509-5516].

FIG. 8: High through-put systems based on RNA/protein interactions. FIG. 8a) shows an allosteric hammerhead ribozyme, which comprises an aptamer component which binds specifically to Rev. When Rev binds to the aptamer component of the ribozyme, the catalytic activity of the ribozyme is reduced. If a suitable FRET substrate is used, which consists in this case of the two components FAM and TAMRA, and the sample is irradiated at 488 nm, there is then energy transfer between FAM and TAMRA, without fluorescence emission. If various compounds, which could, for example, come from a compound library, are added to a reaction mixture containing these components and one of these compounds, small molecules in this case, interacts with Rev, this leads to release of Rev from the aptamer component, resulting in changes in the secondary or tertiary structure of the ribozyme, as a result of which the catalytic activity of the ribozyme is triggered or at least raised. In this case, the FRET substrate is cleaved, and after irradiation at 488 nm, fluorescence emission at 520 nm is generated. This fluorescence emission is the signal which shows that the respective compound, a small molecule in this example, herein also referred to as a candidate molecule, leads to a specific interaction with the target molecule and is accordingly a lead substance for the corresponding target molecule. The Rev protein can be used at 1 μM in this system.

In FIG. 8a), the polynucleotide in accordance with the invention, an allosteric aptazyme, is shown in a procedure in accordance with the invention to identify a compound which interacts with a target molecule. On binding the target molecule, in this case of Rev to the aptamer component of the polynucleotide in accordance with the invention, the ribozyme is present in a catalytically active form. The FRET substrate with FAM and TAMRA, which is bound to the ribozyme and particularly to the catalytically active domain, is cleaved as a result of the catalytic activity, which abolishes the fluorescence quenching, leading to fluorescence emission at 520 nm on irradiation at 480 nm. If one or several compounds to be identified are added to a reaction mixture of this type, and if the or one of these compounds interact with the target molecule, in the present case with the Rev protein, Rev dissociates from the aptamer component of the polynucleotide in accordance with the invention. As a consequence of this, a different pattern of base pairing develops in comparison to the state when the Rev protein was bound to the aptamer component, leading to reduction or suppression of the catalytic activity of the ribozyme. The substrate can no longer be cleaved and there is no longer fluorescence emission, where preferably the substrate can no longer bind to the area of catalytic activity as a result of a change in the pattern of base pairing.

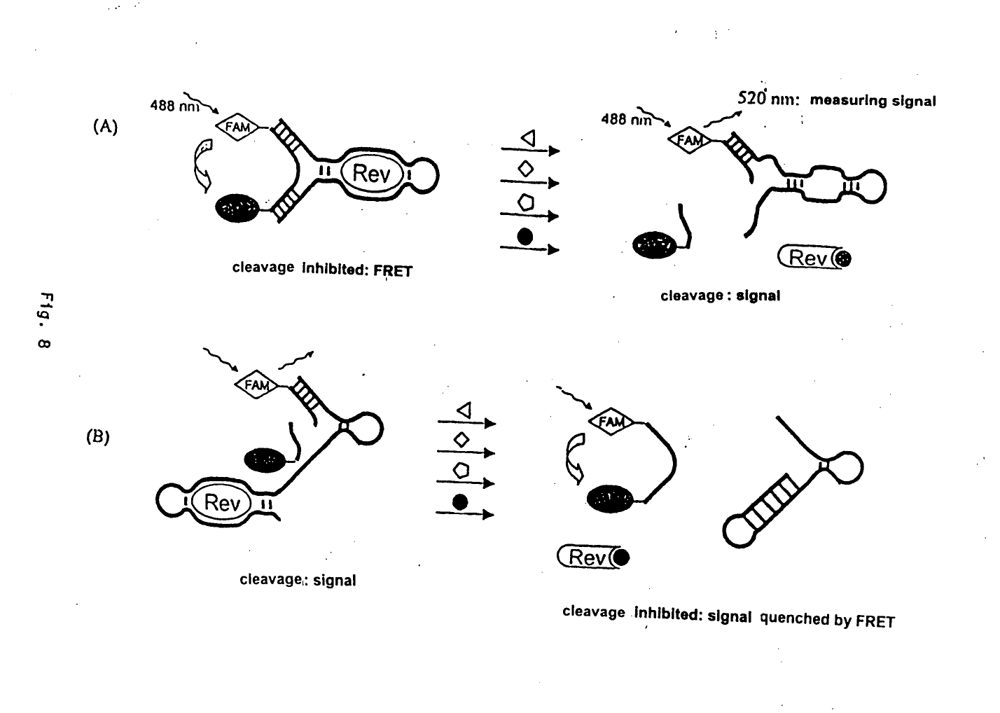

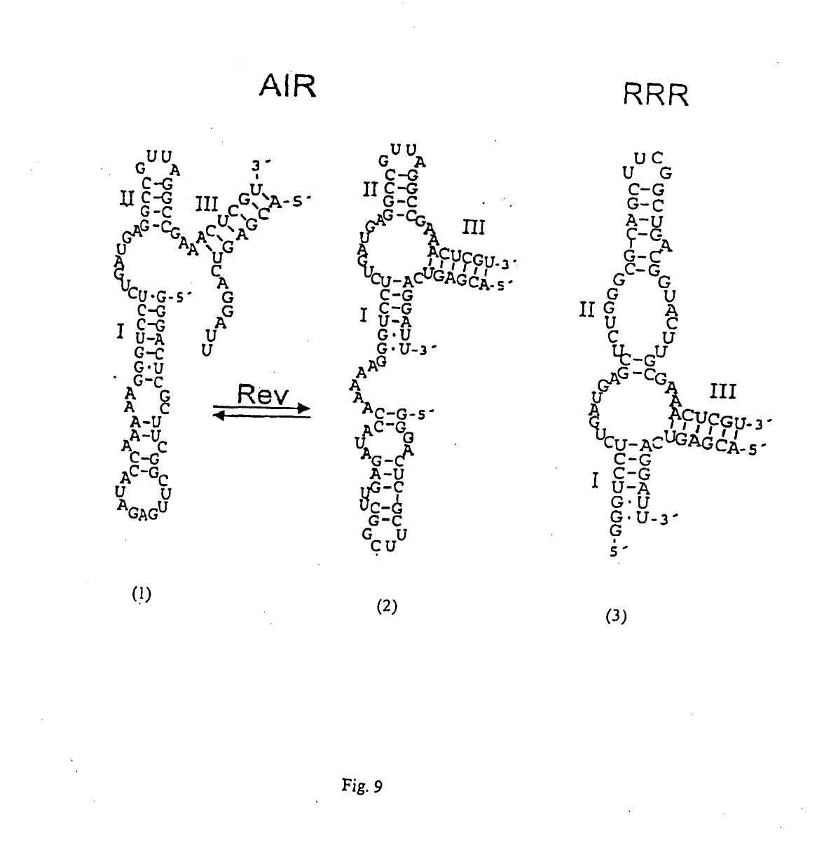

FIG. 9 shows the sequences of the ribozymes in accordance with the invention used for the screening assays described in example 3, which are complexed with a 13mer substrate. The aptamer-inhibited ribozyme AIR consists of a Rev-aptamer, which is bound through a penta-A linker to the 5′-end of the hammerhead ribozyme (HHR), corresponding to a recently discovered hammerhead ribozyme (1) and (2). The aptamer domain forms a helix with the substrate binding site of the ribozyme domain. In the absence of Rev, the aptamer component is not folded, but forms a helix with stem I of the ribozyme and prevents the binding of substrate in this way. After the addition of Rev, the substrate binding site is released and the catalytic reaction (2) can occur. The Rev response ribozyme (RRR) shown as (3) in FIG. 1 contains an HIV genome Rev binding element (RBE) in stem II. RRR is active in the absence of Rev and is inhibited in its presence. The three structures illustrated in FIG. 1 have been folded in accordance with the mfold server algorithm of M. Zucker and show the structures with minimal energy, namely 27.1 and 30.6 kcal/mol for AIR, and 25.4 kcal/mol for RRR. The 3′-ends of the ribozyme and the 5′-ends of the substrate were connected by a tetraloop of GAAA for folding.

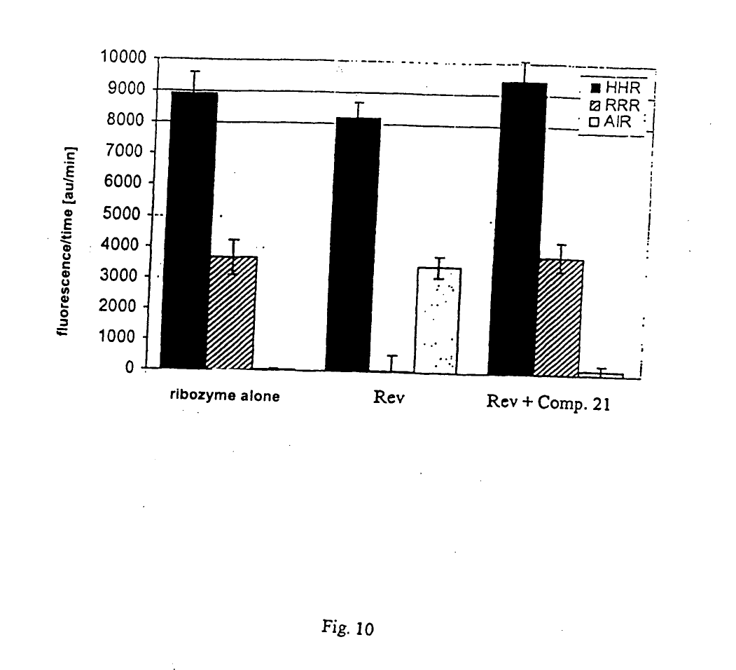

FIG. 10 shows the initial rates of reaction, expressed as measured fluorescence per time for the first five minutes, with the fluorescence of the negative control being subtracted. Illustrated are the reactions with only ribozyme and FRET substrate, Rev reactions, containing 1 μM Rev-peptide in the case of HHR and RRR, 250 nM Rev in the case of AIR and reactions with Rev+compound 21 (Comp. 21), containing Rev and compound 21, coumermycin A1 at 100 μM.

The addition of Rev leads to inhibition of ribozyme RRR and activation of AIR. In the case of RRR and AIR, the action of Rev is fully reversed by 100 μM coumermycin. Wildtype HHR is hardly affected by Rev and compound 21 and serves as internal control.

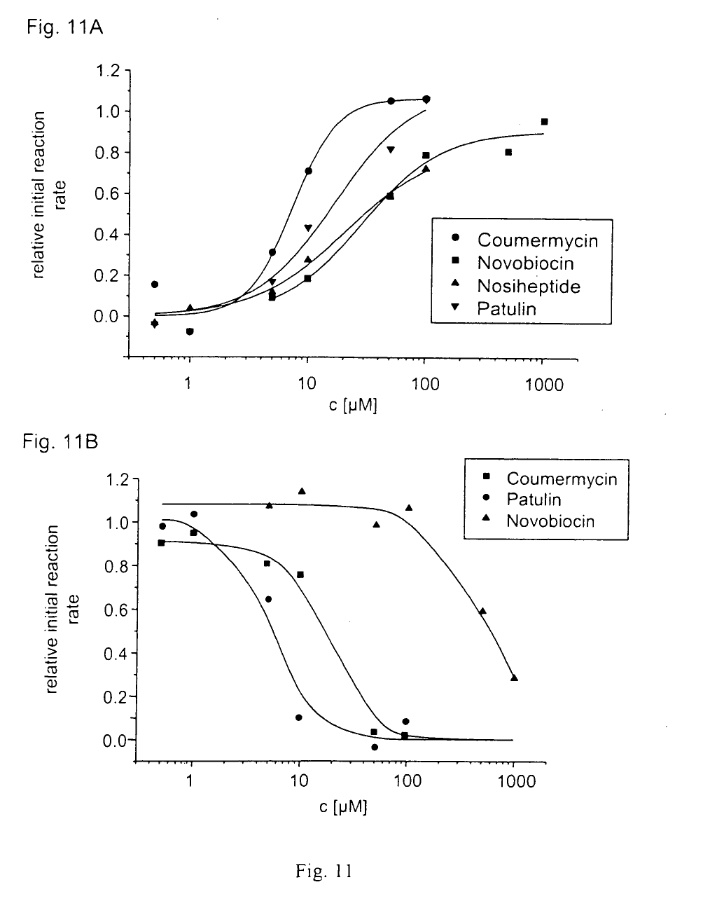

FIG. 11 shows the concentration dependence of the activities of the identified compounds on Rev binding screening ribozymes, RRR and AIR. The initial reaction rates were determined from the initial increase in fluorescence divided by the rate of the ribozyme reaction in the active state in which the compounds were absent. FIG. 11a shows reactions containing 1 μM Rev peptide and various concentrations of coumermycin, novobiocin, nosiheptide and patulin. FIG. 11b shows the AIR reactions which contain 250 nM Rev peptide and various concentrations of coumermycin, novobiocin and patulin.

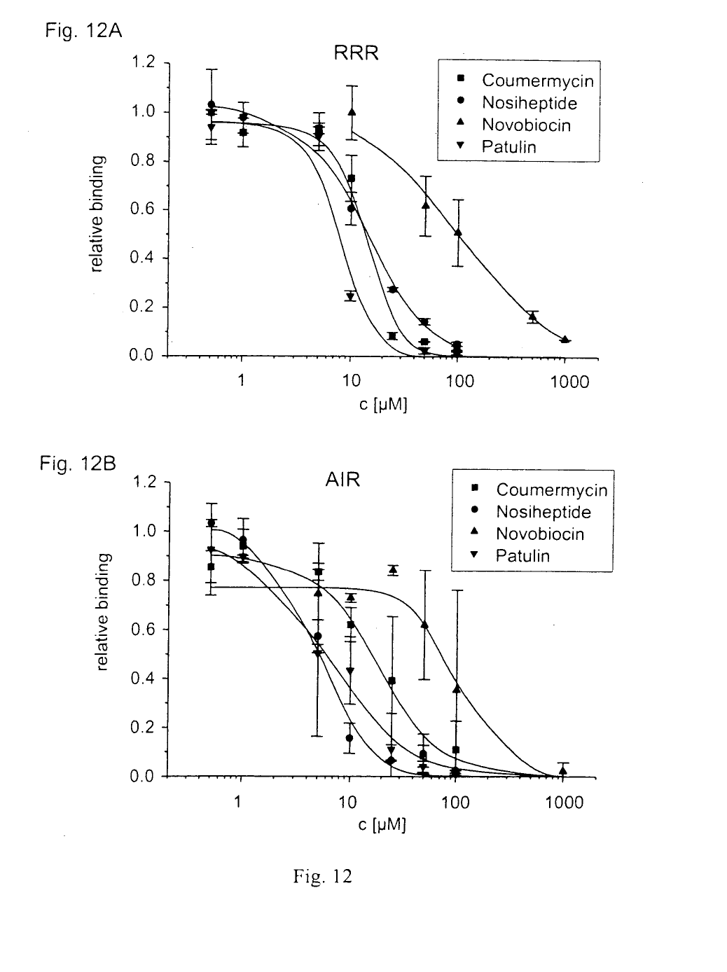

FIG. 12 shows filter binding studies, where the filter binding was performed with 100 nM Rev protein, traces of 5′-³²P-labeled reporter ribozymes, 200 nM FRET substrate and various concentrations of compounds in test buffer at room temperature. The values shown are divided by a control reaction which contains no compound. FIG. 12a shows binding studies with RRR and FIG. 12b binding studies with AIR.

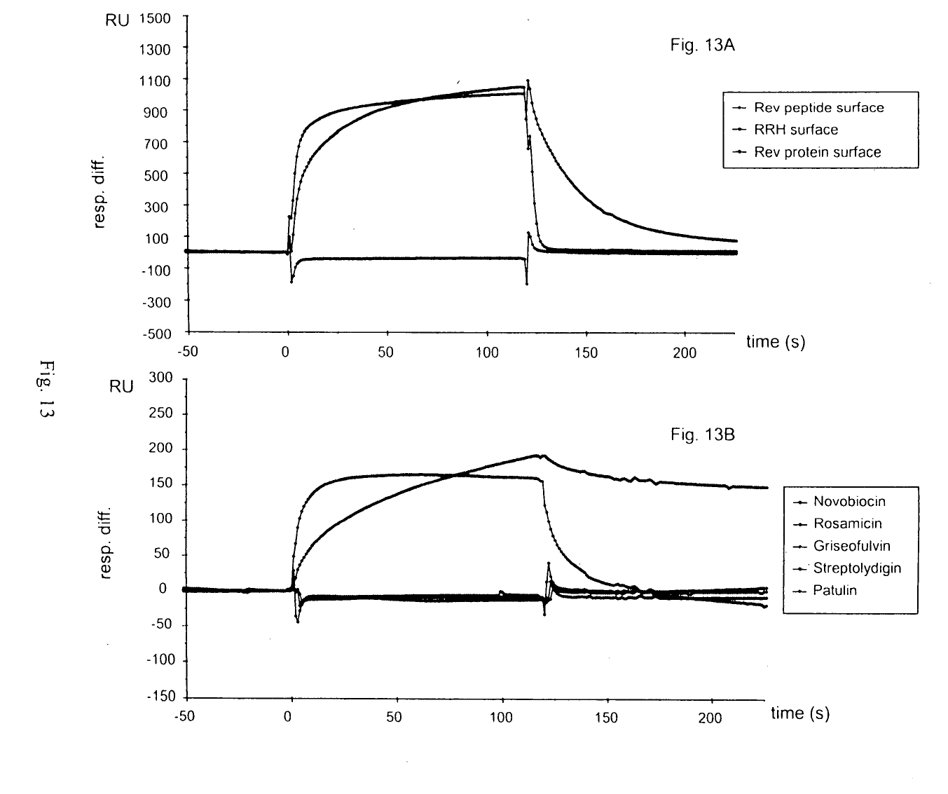

FIG. 13 shows examples for the binding of identified antibiotics to Rev in the context of surface plasmon resonance studies. 20 μl 100 μM antibiotic in test buffer was injected at 25° C., rate 10 μl/s. FIG. 13a shows the binding of coumermycin to surfaces which have been derivatized with Rev peptide and Rev protein. There is no binding to RRR-derivatized surfaces detectable. Coumermycin only binds to Rev, but not to ribozymes. FIG. 13b illustrates the interaction between novobiocin, rosamicin, griseofulvin, streptolydigin and patulin with surfaces derivatized with Rev protein. Only the antibiotics novobiocin and patulin, which were identified in the course of screening, exhibited binding to Rev.

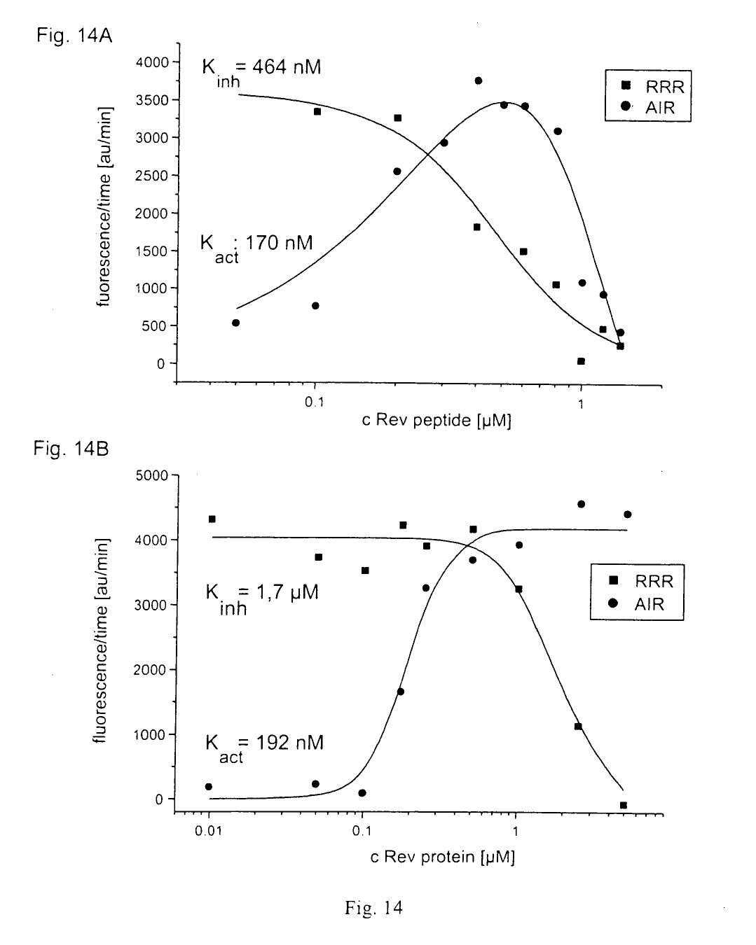

FIG. 14 shows the cleavage activity which was measured as fluorescence per minute in the initial phase (5 min). Negative control reactions without ribozymes were subtracted. FIG. 14a illustrates how the initial cleavage activity for RRR and AIR depends on Rev peptide. HHR is also slightly inhibited by more than 1 μM Rev peptide. FIG. 14b shows how the initial cleavage activity for RRR and AIR depends on Rev protein. HHR is also inhibited at more than 2 μM Rev protein. There was no observed inhibition of AIR by high concentrations of Rev protein, as seen for Rev peptide in FIG. 14a.

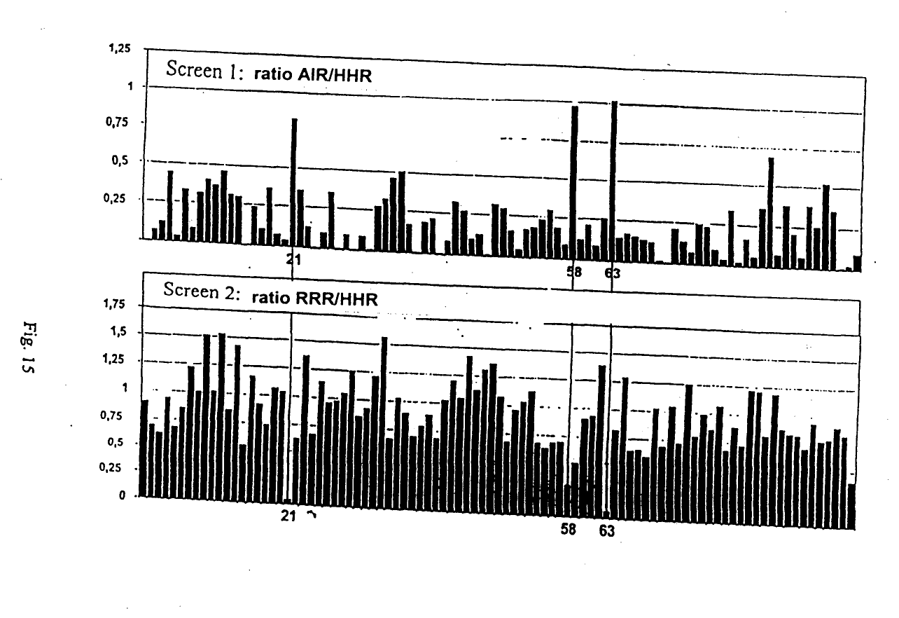

FIG. 15 shows screening results, in which the ratios RRR/HHR and AIR/HHR were generated by subtraction of the fluorescence of the negative control reactions without ribozyme from the initial fluorescence, and division by the values obtained with active states of the ribozymes. Then RRR/AIR values were divided by HHR values, to eliminate general effects on ribozyme function. These reactions contained 10 nM ribozyme, 200 nM substrate, 1 μM (screen 1) or 250 nM (screen 2) Rev peptide and 100 μM antibiotics. As a consequence of the total inhibition, the previously mentioned four most potent inhibitors of hammerhead ribozyme (#8, #31, #91, #92) were removed from the library of 96 antibiotics. In screen 1, Rev effectors were identified by activation of RRR relative to HHR and in screen 2 by inhibition of AIR relative to HHR.

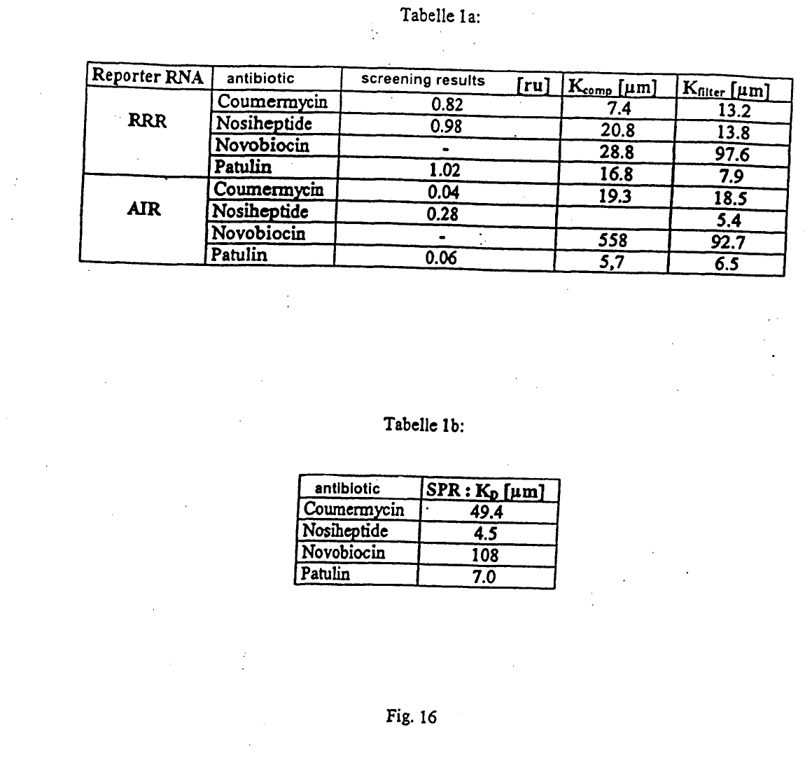

FIG. 16 shows Tables 1a and 1b. Table 1a shows a comparison of the different values determined for selected antibiotics. The column “Screening Results”: Identification with positive (RRR) and negative (AIR) measurements, where the illustrated show initial corrected fluorescence per time, relative to the active state of the ribozyme, and are divided by relative HHR reactions. Novobiocin was not present in the library. K_{comp}: The antibiotic concentrations for half maximal recovery (RRR) or inhibition (AIR) of the cleavage activity obtained by adjusting the data shown in FIG. 11. K_{filter}: The antibiotic concentration for the half maximal release of the ribozyme/Rev protein interaction, obtained by adjusting the data shown in FIG. 12. Table 1b: Kp values determined by surface plasmon resonance for the binding of antibiotics to the Rev protein.

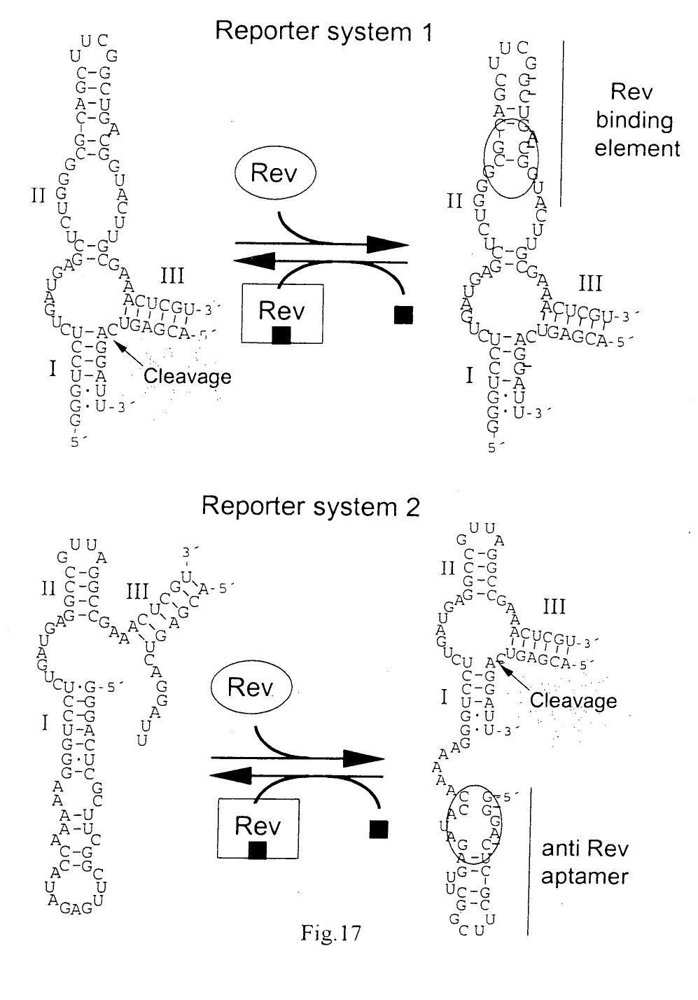

FIG. 17 shows the procedure illustrated in FIG. 8, depicting the secondary structures. The reporter system shown in FIG. 17(a) corresponds to that in FIG. 8(a) and the reporter system in FIG. 17(b) to that in FIG. 8(b).

### EXAMPLE 1

### Screening of Compounds which Bind to the A-Site Subdomain of 16S RNA

With the help of the assay, a library of 500 uncharacterized low molecular weight substance mixtures (molecular weight under 3000 g/mol) was tested on microtiter plates for their binding properties to the target ribozyme construct TRK 1. The mixture of substances was obtained by filtration of bacterial extracts (Actinomycetes strains). TRK1-RNA and the substrate SK1 were produced by automatic solid phase synthesis (for sequences see FIG. 1).

FIG. 2 shows the functional principle of the assay. An inhibitory or activating effect on the cleavage activity of the ribozyme domain was measured as a reduced or increased fluorescence signal, respectively. Table 1 shows a typical result of a screening experiment. FIG. 3 shows a schematic depiction of the assay.

To increase the accuracy of the assay, control reaction mixtures without target ribozyme construct TRK1 were included on the same microtiter plate as the reaction mixture. Subtraction of the fluorescence signals in the control mixtures made it possible to eliminate to a very large extent effects of the substance used on the FRET substrate which were non-specific for the ribozyme or target (e.g. RNA aggregation, quenching effects or RNAse degradation). As shown in FIGS. 4a-c, correction for these non-specific effects clearly reduced the errors in the measurement of the rates of reaction. For example, incubation with substance #332 led to a significant decrease in the fluorescence signal from the control reaction (FIG. 4b). In contrast, an increase in fluorescence was measured in the presence of substance #425, not only in the HHR-1 reaction, but also in the control sample (FIG. 4c).

Two substances from the tested library were identified on the basis of their clear inhibition on the rate of cleavage of the ribozyme domain: substance #122 and substance #387. To exclude the possibility that the observed inhibition or activation was a consequence of the fluorescence measurement procedure, a series of control experiments was performed, in which ribozyme cleavage was examined in a conventional manner with a 5′-³²P labeled substrate (SKU1). This analysis confirmed the results with substances #122 and #387.

In a subsequent control experiment, the ribozyme domain (HHR1) of the TRK1 was examined separately, i.e. without linker sequence and 16S RNA domain, in identically performed experiments. The missing sequence was then replaced by the hairpin structure 5′-CCGGAUUGCCGG-3′. It was shown that substance #122 inhibits ribozyme HHR1, while substance #387 has no measurable effect on the ribozyme. A binding study with the 16S domain shown in FIG. 1 (Biacore analysis) confirmed that substance #122 binds specifically and with high affinity to the A-site subdomain.

Table 1: Typical results of a screening experiment (substances No 97-No192). The table shows the relative activity of the reporter ribozyme in the presence of 100 μM of each substance. The values were obtained by regression analysis, followed by normalization, where the initial rate for the non-inhibited reaction was set “1”. Substances are highlighted where there was a clearly inhibitory effect. All tests were performed with substrate excess, with 8 nM TRK1, 200 nM SK1 in 0.5×PBS at 32° C., with 8 mM MgCl₂. The reactions were started by simultaneous addition of MgCl₂ and substance.

Table 1: Typical results of the screening experiment (Substances 97-197).

### EXAMPLE 2

### Screening of Protein-Binding Substances by Using an Intramer-Ribozyme Construct

Screening for a substance which binds to the viral Rev protein was performed on a library of 50 short RNA sequences, using the procedure in accordance with the invention. The sequences are as follows:

Seq 1:
5′-GGGAGUUGAUAACAGGCUCAAUGAGCCUGCUCGGUCAAC -3′
Seq 2:
5′-GGGAGUUGAUAGCAGGCUCAAUGAGCCUGAGUUCCCAAC -3′
Seq 3:
5′-GGGAGUUGAUAUCAGGCUCAAUGAGCCUGGUCGACCAAC -3′
Seq 4:
5′-GGGAGUUGAUACCAGGCUCAAUGAGCCUGCAAAGUCAAC -3′
Seq 5:
5′-GGGAGGUGGACUCCAGCUUCGGCUGUUGAGAUACACC-3′
Seq 6:
5′-GGGAGUUGGUACCAGGCUCAAUGAGCCUGAAAGCUCAAC -3′
Seq 7:
5′-GGGAGUUGCUACCAGGCUCAAUGAGCCUGGUUAAACAAC -3′
Seq 8:
5′-GGGAGUUGUUACCAGGCUCAAUGAGCCUGCGCGCGCAAC -3′
Seq 9:
5′-GGGAGUUGUAACCAGGCUCAAUGAGCCUGUAUAUACAAC -3′
Seq 10:
5′-GGGAGUUGUGACCAGGCUCAAUGAGCCUGAGAAUCCAAC -3′
Seq 11:
5′-GGGAGUUGUCACCAGGCUCAAUGAGCCUGCCUGGACAAC -3′
Seq 12:
5′-GGGAGUUGGCAUCAGGCUCAAUGAGCCUGUGGACACAAC -3′
Seq 13:
5′-GGGAGUUGGACUCAGGCUCAAUGAGCCUGGAAAAACAAC -3′
Seq 14:
5′-GGGAGUUGGAAUCAGGCUCAAUGAGCCUGAGGGGACAAC -3′
Seq 15:
5′-GGGAGUUGGCCUCAGGCUCAAUGAGCCUGCUUUUCCAAC -3′
Seq 16:
5′-GGGAGUUGGAAUCAGGCUCAAUGAGCCUGUCCCCUCAAC -3′
Seq 17:
5′-GGGAGUUGGCCUCAGGCUCAAUGAGCCUGACCCCACAAC -3′
Seq 18:
5′-GGGAGUUGGGGUCAGGCUCAAUGAGCCUGGUUUUGCAAC -3′
Seq 19:
5′-GGGAGUUGGUUUCAGGCUCAAUGAGCCUGCGGGGGCAAC -3′
Seq 20:
5′-GGGAGUUGAAUUCAGGCUCAAUGAGCCUGUAAAAUCAAC -3′
Seq 21:
5′-GGGAGUUGAACCCAGGCUCAAUGAGCCUGAUAUAUCAAC -3′
Seq 22:
5′-GGGAGUUGAACACAGGCUCAAUGAGCCUGUAUAUACAAC -3′
Seq 23:
5′-GGGAGUUGAAGGCAGGCUCAAUGAGCCUGAAAUUUCAAC -3′
Seq 24:
5′-GGGAGUUGAAGUCAGGCUCAAUGAGCCUGUUUAAACAAC -3′
Seq 25:
5′-GGGAGUUGAGUCCAGGCUCAAUGAGCCUGAAUUAACAAC -3′
Seq 26:
5′-GGGAGUUGUUUCCAGGCUCAAUGAGCCUGUUAAUUCAAC -3′
Seq 27:
5′-GGGAGUUGGGGGCAGGCUCAAUGAGCCUGCCCAAACAAC -3′
Seq 28:
5′-GGGAGUUGGCAACAGGCUCAAUGAGCCUGCACAGUCAAC -3′
Seq 29:
5′-GGGAGUUGACCCCAGGCUCAAUGAGCCUGGUGCAGCAAC -3′
Seq 30:
5′-GGGAGUUGCACACAGGCUCAAUGAGCCUGGUCAGCCAAC -3′
Seq 31:
5′-GGGAGUUGCAUACAGGCUCAAUGAGCCUGCAGUUACAAC -3′
Seq 32:
5′-GGGAGUUGUGAGCAGGCUCAAUGAGCCUGGGCAGUCAAC -3′
Seq 33:
5′-GGGAGUUGGGUCCAGGCUCAAUGAGCCUGUCAACUCAAC -3′
Seq 34:
5′-GGGAGUUGCGAUCAGGCUCAAUGAGCCUGACUAGGCAAC -3′
Seq 35:
5′-GGGAGUUGAAUCCAGGCUCAAUGAGCCUGUUGCACCAAC -3′
Seq 36:
5′-GGGAGUUGUCGGCAGGCUCAAUGAGCCUGACGUACCAAC -3′
Seq 37:
5′-GGGAGUUGUCCGCAGGCUCAAUGAGCCUGUGGUAACAAC -3′
Seq 38:
5′-GGGAGUUGAGAACAGGCUCAAUGAGCCUGCUCCGACAAC -3′
Seq 39:
5′-GGGAGUUGCCUCCAGGCUCAAUGAGCCUGACCUCGCAAC -3′
Seq 40:
5′-GGGAGUUGCCCGCAGGCUCAAUGAGCCUGGCAGCCCAAC -3′
Seq 41:
5′-GGGAGUUGGCGCCAGGCUCAAUGAGCCUGUCGAAGCAAC -3′
Seq 42:
5′-GGGAGUUGCGCGCAGGCUCAAUGAGCCUGUCACACCAAC -3′
Seq 43:
5′-GGGAGUUGCGAACAGGCUCAAUGAGCCUGAAAAAACAAC -3′
Seq 44:
5′-GGGAGUUGUCUUCAGGCUCAAUGAGCCUGUUUUUUCAAC -3′
Seq 45:
5′-GGGAGUUGGGAGCAGGCUCAAUGAGCCUGGGGGGGCAAC -3′
Seq 46:
5′-GGGAGUUGGCCUCAGGCUCAAUGAGCCUGCCCCCCCAAC -3′
Seq 47:
5′-GGGAGUUGGAUGCAGGCUCAAUGAGCCUGAAAUGGCAAC -3′
Seq 48:
5′-GGGAGUUGCCCUCAGGCUCAAUGAGCCUGAAAUGGCAAC -3′
Seq 49:
5′-GGGAGUUGCUCUCAGGCUCAAUGAGCCUGAUAUGGCAAC -3′
Seq 50:
5′-GGGAGUUGCUCGCAGGCUCAAUGAGCCUGAAAUGACAAC -3′

The DNA matrixes coding for the 50 RNA sequences were produced by in vitro transcription of oligonucleotides synthesized by solid phase synthesis. After transcription, the RNAs were separated by length, using denaturing polyacrylamide gel electrophoresis in denaturing urea/polyacrylamide gels. The corresponding bands were then visualized by fluorescence quenching. For this purpose, the gels were packed in transparent foil, laid on DC aluminium foils (Kieselgel 60 F245, Merck) and irradiated with a pocket lamp at 254 nm. Bands of the right length were cut out and the slices of gel were cut up into small pieces and covered with 300 mM sodium acetate (pH 5.2). After incubation for 1 h at 65° C. and for 4 h at room temperature, the gel suspension was pressed through a syringe which was filled with glass wool. After removing gel residues, the nucleic acids were precipitated and taken up in sterile water.

For the screening experiment, a binding RNA sequence (cf. FIG. 7) for the Rev protein of HIV was isolated from the library of 50 different ribonucleic acids. FIG. 5 shows how the assay functions in principle. The experiment was performed with the sequences IRK1 and SK1 shown in FIG. 6, analogously to Example 1. The two sequences were produced by oligonucleotide solid phase synthesis and were purchased from Eurogentec (Belgium). FIG. 7 shows a general formula for the sequences which do not bind to the Rev protein and for the identified Rev-binding RNA sequence. The experiment was performed as follows: The sequences from the library and the IRK-1 were denatured separately in reaction buffer (10 mM HEPES (pH 7.4), 100 mM NaCl) for 1.5 min at 95° C. and then cooled at room temperature to renature. The IRK-1 and SK-1 were mixed and incubated for 5 min at room temperature. This incubation mixture was then divided equally between 50 individual reactions. Rev protein was added to the reaction mixtures, which were then incubated for a further 15 min at room temperature. The 50 RNA sequences from the library were then added to the 50 reaction mixtures. After another 30 min, the cleavage reaction for substrate SK-2 was started by the addition of magnesium. The final concentrations in the reaction mixtures were as follows: 10 mM HEPES (pH 7.4), 100 mM NaCl, 10 mM MgCl₂, IRK-18 nM, SK-1200 nM, RNA sequences from the library 2 μM, Rev protein 200 nM. All components were prewarmed to 37° C. before mixing. The evaluation was carried out as in Example 1 and led to the identification of the sequence in FIG. 7 as a Rev-binding sequence, which could compete with IRK-1 for binding to the protein. The identification was made on the basis of the marked differences in fluorescence in comparison with the reaction mixtures from the other members of the library. This experiment demonstrated that competitive inhibitors of an RNA/protein interaction can be easily isolated from combinatorial libraries using the procedure in accordance with the invention and fluorescence detection which is compatible with HTS procedures.

### EXAMPLE 3

### Assembly of a High-Throughput Screening System for the Identification of Compounds which Interact with the Target Protein Rev

The data from this example are also the object of FIGS. 8 to 17.

The interaction between the Rev protein of HIV and RRE facilitates the export of unspliced viral RNA, which is important for viral replication and which is therefore the object of many approaches to the development of antiviral therapies [Dayton, A. I. & Zhang, M. J., Therapies directed against the Rev axis of HIV autoregulation, Adv Pharmacol 49, 199-228 (2000); Pollard, V. W. & Malim, M. H., The HIV-1 Rev protein, Annu Rev Microbiol 52, 491-532 (1998)]. In the context of the work described here, two different hammerhead ribozymes were constructed; their secondary structure is shown in FIG. 9 and their basic mode of action in FIG. 8. The ribozyme which can be activated by Rev is referred to as aptamer-inhibited ribozyme (AIR). This was constructed by fusion of the Rev aptamer [Symensma, T. L., Giver, L., Zapp, M., Takle, G. B. & Ellington, A. D., RNA aptamers selected to bind human immunodeficiency virus type 1 Rev in vitro are Rev responsive in vivo, J Virol 70, 179-87 (1996)] at the 5′-end of the ribozyme, using a penta-A linker. The second ribozyme was inhibited by Rev. This was constructed by inserting the high affinity Rev-binding element (RBE) of the RRE HIV genomic sequence into stem II of the hammerhead ribozyme. This yielded the R ev-responsive ribozyme (RRR) (cf. FIGS. 8 and 9).

Ribozyme Reactions:

Cleavage reactions with multiple turnover were performed at 32° C. in a test buffer containing 50 mM Tris (pH 7.9) and 25 mM NaCl. The total volume of the reactions was 50 μl in plates with 96 flat bottom wells. The fluorescence measurements were performed at an excitation wavelength of 489 nm, FAM emission at 520 nm and with the fluorescence detector Ascent Fluoroscan FL, as described by Jenne, A. et al., Rapid identification and characterization of hammerhead-ribozyme inhibitors using fluorescence-based technology, Nat Biotechnol 19, 56-61 (2001). Ribozyme (10 nM) and FRET-labeled substrate (200 nM) were first incubated in test buffer at room temperature for 5 min, followed by optional addition of Rev and/or antibiotic. The reactions were then incubated for 5 min at 32° C. and started by the addition of MgCl₂ to a final concentration of 8 mM, using the dispensing function of the fluorometer. Negative controls were always included for reactions without ribozyme and were subtracted after the measurements.

Filter Binding Studies:

Reaction mixtures (20 μl) containing 100 nM Rev protein, traces of 5′-³²P labeled RRR or AIR, 200 nM FRET substrate and various concentrations of antibiotic in test buffer were washed through a pre-rinsed 0.45 μm nitrocellulose membrane, followed by washing with 3 ml test buffer. The sequence of additions and incubation times was performed as with the ribozyme reactions.

Surface Plasmon Resonance:

Surface plasmon resonance studies were performed with a Biocore 3000 instrument in the automatic mode. Rev protein and peptide surfaces were prepared by injection of 100 μM solutions in 60 mM NaOAc, pH 5.7 on EDC/NHS-activated CM5-Chips, which gave 1800 RU immobilized peptide and 5000 RU protein. The RRR-RNA surface was produced by the injection of biotinylated RRR (75 nM) in Tris pH 7.5, 0.5 M NaCl on a streptavidin-derivatized chip and gave 1600 RU immobilized RRR.

Result:

Both new apatazymes, AIR and RRR, are regulated by the RNA domain for the Rev protein, the intact Rev protein and by the Rev peptide, which includes aminoacids 34 to 50 of the Rev protein. The surface plasmon resonance procedure gave Kp values as follows: 1.4 nM for the RRR/Rev protein, 9.3 nM for the RRR/Rev peptide, 1.3 nM for the AIR/Rev protein, and 9.0 nM for the AIR/Rev peptide.

The kinetic characterization of the new hammerhead ribozyme constructs under the conditions of multiple turnover showed that the initial reaction rate for the Rev-regulated RRR was 38% of the reaction rate of a well characterized hammerhead ribozyme, HHR (Jenne, A., Gmelin, W., Raffler, N. & Famulok, M., Real-Time Characterization of Ribozymes by Fluorescence Resonance Energy Transfer (FRET), Angew. Chem. Int. Ed. 38, 1300-1303 (1999); Jenne, A. et al., Rapid identification and characterization of hammerhead-ribozyme inhibitors using fluorescence-based technology, Nat Biotechnol 19, 56-61 (2001). In contrast to this, the initial rate with the aptamer-inhibited ribozyme AIR was only 1.4% of that with HHR (cf. FIG. 11). After adding 1 μM Rev peptide, RRR gave only 1.0% of the activity of HHR, corresponding to inhibition by a factor of 36. In contrast, the addition of 250 nM Rev peptide to ribozyme AIR gave 32-fold activation, to a level of 44% of the activity of HHR.

The inhibition of RRR at the half maximal concentration of Rev: K inh=464 for Rev peptide (cf. FIG. 14a) and K inh=1.7 μM for Rev protein (cf. FIG. 14b).

The activation of AIR: K_{act}=170 nM for Rev peptide (cf. FIG. 14a) and K act=192 nM for the Rev protein (cf. FIG. 14b). At Rev peptide concentrations of above 800 nM, AIR is inhibited as well (cf. FIG. 14a). This effect can be explained as unspecific inhibition of the catalytic ribozyme function by the strongly positively charged peptide. Even HHR is inhibited at Rev concentrations of above 1 μM. As shown in FIG. 11, the initial activity is 93% at a concentration of 1 μM, although the cleavage is inhibited after some minutes. When the Rev protein is used, AIR is still activated at concentrations of above 5 μM, whereas RRR is inhibited above 1 μM and HHR above 2 μM Rev protein (cf. FIG. 14b).

Screening of 96 Antibiotics:

Two different screening experiments were performed, one using the RRR reporter construct and the other using AIR. The following standard reactions in duplicate were prepared for each plate with 96 wells: ribozyme (HHR and reporter construct) alone, ribozyme reactions containing Rev peptide (1 μM in the case of RRR screening, 0.25 μM in the case of AIR screening), two negative controls without ribozyme, the first only with substrate, and the second with Rev and substrate. Each reaction which contained antibiotics also contained Rev peptide. For each antibiotic, three different reactions were performed: HHR, reporter construct (RRR or AIR), negative controls, which only contained antibiotic and Rev and substrate. The antibiotic concentrations were 100 μM in each case. After subtraction of the negative control, the rise in fluorescence in the first five minutes, corresponding to the initial rate of reaction, was divided by the rise in fluorescence in the standard reaction without antibiotic, containing the active state of the ribozyme (see also FIG. 10). In order to only describe specific effects on the Rev RNA interactions and not on the function of the hammerhead, the values obtained for the RRR and AIR were divided by the values for the HHR reactions.

In all, two screens were performed and the results of these are shown in FIG. 15. RRR inhibited by 1 μM Rev peptide was used for one, where active compounds were identified by the restoration of the cleavage catalytic activity of the ribozyme. In the second screen, the ribozyme AIR was used; compounds which are capable of preventing the interaction between Rev and the Rev aptamer are identified on the basis of the inhibition of the Rev reporter construct, which is activated by 250 nM Rev peptide. The most active inhibitors of the hammerhead ribozyme, antibiotics #8, #31, #91 and #92, could not be examined, as they inhibited the reaction completely.

Inactive compounds give readings of about zero in the RRR screen, because of the inhibition by Rev. Active compounds give reactivation of the reporter construct RRR; total reactivation gives a reading of 1. Three compounds were identified which gave significant activation (above 0.75), namely coumermycin A1 (#21), nosiheptide (#58) and patulin (#63), as also shown in Table 1a.

In the AIR screen, inactive compounds give a reading of about 1, as they leave the reporter construct in its active Rev-binding state. Active compounds gave low values near to zero and were identified on this basis (total inhibition). It was striking that screening of the compound library of 96 antibiotics led to the identification of the same compounds (#21, #58 and #63), confirming that these are Rev inhibitors.

FIG. 11 shows the results of the measurement of the concentration dependence of the abolition of the interaction between Rev and the ribozyme. Novobiocin, a natural analog of coumermycin A, was included in all further studies. The results of the studies of relative initial rates are summarized in Table 1a.

Characterization of the Identified Antibiotics

Conventional filter binding studies were performed as a further study of the interactions observed in fluorescence measurements. Rev protein rather than Rev peptide was used for this purpose, as the peptide is too small to be retained by the filter. 50-60% of the 5′-³²P-labeled reporter ribozymes RRR and AIR were retained on the filter in the presence of 100 nM Rev protein. The addition of the identified antibiotics confirms once again that these antibiotics are capable of interfering with the interaction between RNA and the protein (cf. FIG. 12). It is very striking that these Rev protein binding experiments confirm the results obtained by fluorescence measurements with the Rev peptide.

The concentrations of the compounds at which 50% of the RNA protein complexes are dissociated are summarized in Table 1a.

Surface plasmon resonance was used for a more exact clarification of the interaction between the three components used in the test. The binding of the antibiotics was tested on surfaces which had been derivatized with RRR-RNA, Rev peptide and with the Rev protein. It was interesting that all four antibiotics demonstrated binding to both the Rev peptide and the Rev protein, whereas there was no detectable interaction for various other compounds in the library which had not been identified in the screening. The four Rev-binding antibiotics exhibited no binding to RRR-RNA. The K_{D} values for the binding of the antibiotics are given in Table 1b. With the exception of coumermycin, these values agree closely with the apparent binding constants determined by filter binding and fluorescence measurements.

As a control of the target protein reverse transcriptase, another nucleic acid binding protein from HIV-1 was tested, to check the specificity of the identified Rev-binding antibiotics. No significant effects were observed at antibiotic concentrations of 100 μM in a standard transcriptase assay (while ddCTP gave total inhibition at 2.5 μM).

The conclusion from these results is therefore that the identified antibiotics and, in general, compounds identified by procedures in accordance with the invention, can be used as lead substances for future pharmaceutically active substances.

The features of the invention, as disclosed in the above description and in the claims and drawings may, either individually or in any combination, be essential for the implementation of the invention in its different embodiments.

### SUMMARY

Described is a procedure for the identification of compounds (lead structures) which (a) bind specifically to a desired RNA target motif and which can, as a result, inhibit or eliminate the function of this or which (b) can displace a compound which is associated with a desired RNA target motif and which can as a result inhibit or eliminate the function of this or these. The procedure in accordance with the invention is based on the binding of a ligand (=compound to be identified) to an RNA target motif which is coupled to a modified ribozyme, so that the ribozyme is converted to an active or to an inactive conformation, leading to the cleavage of a signaling ribozyme substrate. The compounds identified in this way permit the modification of the cellular function of the RNA target motifs and permit the production of specific drugs. The invention also relates to a polynucleotide comprising a hammerhead ribozyme and an aptamer for the target molecule, where there is a difference in the base pairing pattern of the polynucleotide when the target molecule is bound to the aptamer and when the target molecule is absent from the aptamer.

1. A method for identifying compounds which bind specifically to a desired RNA-target motif and, as a result, can inhibit or eliminate the function of this and which has the following characteristics:
(a) Generation of a construct (target reporter construct; TRK) from a reporter ribozyme domain (I) and the RNA target motif (II), where (I) and (II) are linked to each other by an RNA linker and where the reporter ribozyme domain (I) changes its catalytic activity after specific binding of a compound to the RNA target motif (II);
(b) Generation of a signaling ribozyme substrate which can specifically bind to the reporter ribozyme domain (I);
(c) Bringing into contact of the TRK from step (a) and the ribozyme substrate from step (b) with the compound to be identified or a mixture which contains this compound; and
(d) Determination of the binding of the compound to the RNA target motif.

2. A method for identifying compounds which can displace a compound that is associated with a desired RNA target motif and which, as a result, can inhibit or eliminate the function of this, being characterized by the following steps:
(a) Production of a construct (target reporter construct; TRK) from a reporter ribozyme domain (I) and the RNA target motif (II), where (I) and (II) are linked to each other by an RNA linker and where the reporter ribozyme domain (I) alters its catalytic activity after displacement of the compound associated with the desired RNA target motif from the RNA target motif (II);
(b) Production of a signaling ribozyme substrate which specifically binds to the reporter ribozyme domain (I);
(c) Bringing into contact of the TRK from step (a) with the compound which is naturally associated with the RNA target motif;
(d) Bringing into contact of the complex from step (c) and the ribozyme substrate from step (b) with the compound which is to be identified or a mixture which contains this compound; and
(e) Determination of the displacement of the compound associated with the RNA target motif.

3. The method according to claims 1 or 2, in which the ribozyme substrate can be cleaved by the reporter ribozyme domain (I) in step (b) and in which the binding is determined in the last step by the cleavage of the ribozyme substrate.

4. The method according to any of claims 1 to 3, in which the reporter ribozyme domain originates from a hammerhead ribozyme.

5. The method according to any of claims 1 to 4 in which the ribozyme substrate is a doubly labeled ribozyme substrate.

6. The method according to any of claims 1 to 5, in which the doubly labeled ribozyme substrate contains a fluorophore group and a fluorescence quenching group and in which the quenching of the fluorescence of the fluorophore by the fluorescence quenching group is prevented after cleavage of the reporter ribozyme domain.

7. The method according to claim 6, in which the fluorophore group is 6-carboxyfluorescein (FAM) and the fluorescence quenching group is 6-carboxytetramethylrhodamine (TAMRA).

8. A drug containing a compound which has been identified by a method according to any of claims 1 to 6.

9. A kit for the performance of a method according to any of claims 1 to 9, where the kit comprises the following compounds:
(a) A target reporter construct; and
(b) A signaling ribozyme substrate.

10. A polynucleotide comprising a hammerhead ribozyme and an aptamer for a target molecule, where the binding site of the catalytic domain of the ribozyme for a ribozyme substrate is blocked to the binding of the substrate of the ribozyme in the absence of the target molecule of the aptamer.

11. The polynucleotide comprising a hammerhead ribozyme and an aptamer for a target molecule, in particular according to claim 10, which also comprises the target molecule bound to the aptamer, where the binding site of the catalytic domain of the ribozyme for a ribozyme substrate is accessible for the binding of a substrate for the ribozyme in the presence of the target molecule of the aptamer.

12. The polynucleotide comprising a hammerhead ribozyme and an aptamer for a target molecule, in particular a polynucleotide according to claims 10 or 11, in which the pattern of base pairing differs, depending on whether the target molecule of the aptamer is bound or absent.

13. The polynucleotide according to any of claims 10 to 12 which also comprises a substrate for the catalytic activity of the ribozyme.

14. The polynucleotide according to claim 13, characterized by the substrate being an FRET substrate.

15. The polynucleotide according to any of claims 10 to 12, comprising a nucleic acid sequence which is selected from the group comprising SEQ. ID. No. 51 and SEQ. ID. No 52.

16. The polynucleotide according to any of claims 10 to 15, characterized by the polynucleotide being RNA, DNA or mixtures of these.

17. A biosensor, comprising a polynucleotide according to any of claims 10 to 16.

18. The biosensor according to claim 17, characterized by the polynucleotide being bound to a solid carrier.

19. A method for identifying a compound which binds to a target molecule, including the following steps:
a) Providing a polynucleotide according to any of claims 10 to 16,
b) Optionally determining the catalytic activity of the ribozyme,
c) Adding the target molecule, where the target molecule interacts with the nucleic acid which binds the target molecule,
d) Optionally determining the catalytic activity of the ribozyme,
e) Adding a candidate compound,
f) Optionally determining the catalytic activity of the ribozyme,
g) Providing a substrate for the catalytic activity of the polynucleotide and adding of the substrate to the reaction mixture,
h) Determining the binding of the candidate compound to the target molecule.

20. The method according to claim 19, being characterized by that either before step f) or after step g), the candidate compound is submitted to a procedure, comprising the steps:
a) Providing a polynucleotide, whereby the polynucleotide is an allosteric ribozyme and comprises a hammerhead ribozyme and an aptamer which is specific for the target molecule, preferably the aptamer of the polynucleotide according to claim 19,
b) Optionally determining the catalytic activity of the ribozyme,
c) Adding the target molecule, whereby the target molecule interacts with the nucleic acid which binds the target molecule,
d) Optionally determining the catalytic activity of the ribozyme,
e) Addition of the candidate compound,
f) Optionally determining the catalytic activity of the ribozyme,
g) Providing a substrate for the catalytic activity of the polynucleotide and adding the substrate to the polynucleotide, and
h) Determining the binding of the candidate compound to the target molecule.

21. The method according to claim 19 or 20, characterized by the determination of the binding of the candidate molecule to the target molecule being performed by determining the enzymatic activity of the ribozyme.

22. The method according to claim 21, characterized by the substrate containing a fluorophore group and a fluorescence quenching group and the cleavage of the substrate by the catalytic activity of the ribozyme or the catalytic domain preventing or at least reducing the quenching of the fluorescence.

23. The method according to claim 22, characterized by the fluorophore group being 6-carboxyfluorescein and the fluorescence quenching group being 6-carboxytetramethyl rhodamine or Cy 3.

24. A drug containing a compound which was identified according to any of claims 19 to 23.

25. A kit for the performance of a procedure according to any of claims 19 to 23, comprising
a) A polynucleotide in accordance with one of claims 10 to 16; and
b) A signaling ribozyme substrate.

26. Use of coumermycin for the production of a drug for the treatment of HIV and/or FIV.

27. Use of nosiheptide for the production of a medicine for the treatment of HIV and/or FIV.

28. Use of patulin for the production of a medicine for the treatment of HIV and/or FIV.
